(19) United States
(12) Patent Application Publication
(10) Pub. No.: US 2025/0191752 A1
(43) Pub. Date: Jun. 12, 2025

(54) SYSTEMS AND METHODS FOR AUTOMATED DETERMINATION OF A PROSTATE CANCER STAGING SCORE

(71) Applicants: Progenics Pharmaceuticals, Inc., N. Billerica, MA (US); Exini Diagnostics AB, Lund (SE)

(72) Inventors: Karl Vilhelm Sjöstrand, Atlantic Highlands, NJ (US); Kamil Rzechowski, Warsaw (PL)

(21) Appl. No.: 18/667,945

(22) Filed: May 17, 2024

Related U.S. Application Data
(60) Provisional application No. 63/606,824, filed on Dec. 6, 2023.

(51) Int. Cl. G16H 50/20; G16H 30/40

(52) U.S. Cl. CPC G16H 50/20; G16H 30/40

(57) ABSTRACT

Presented herein are systems and methods for the automated determination of a prostate cancer staging score (e.g., a PRIMARY score) for a subject. In certain embodiments, the systems and methods employ a machine learning model (e.g., one or more convolutional neural networks, CNNs) to analyze three-dimensional (3D) images obtained via both a functional imaging modality and an anatomical imaging modality. In addition to identifying regions of PSMA binding agent uptake (hotspots), the techniques described herein are able to accurately and automatically associate specific prostate zones to each hotspot and use this information in the determination of the staging score.

### CROSS-REFERENCE TO RELATED APPLICATION

This application claims priority to and benefit from U.S. Provisional Application No. 63/606,824, filed Dec. 6, 2023, the content of which is hereby incorporated by reference in its entirety.

### FIELD

This invention relates generally to systems and methods for analysis of medical images. More particularly, in certain embodiments, the present disclosures provides systems and methods for automated, machine learning based determination of a prostate cancer staging score from functional and anatomical medical images.

### BACKGROUND

Prostate-specific membrane antigen (PSMA)-targeted positron emission tomography (PET) has recently been used for staging of patients with prostate cancer. In particular, the PSMA binding agents ⁶⁸Ga-PSMA-11 (gallium (⁶⁸Ga) gozetotide, e.g., Illucix®) and [¹⁸F]DCFPyL (piflufolastat F 18, e.g., PYLARIFY®) were approved by the U.S. Food and Drug Administration in 2020 and 2021, respectively, and there is a growing body of evidence that supports integration of PSMA-PET into clinical guidelines.

Patterns of PSMA expression in the prostate can be characterized from PSMA-PET images using a scoring system referred to as a PRIMARY score, described, for example, in Ceci et al., “The EANM Standardized Reporting Guidelines v1.0 for PSMA-PET,” Eur J Nucl Med Mol Imaging 2021; 48: 1626-38, Seifert et al., “Second Version of the Prostate Cancer Molecular Imaging Standardized Evaluation Framework Including Response Evaluation for Clinical Trials (PROMISE V2),” European Urology 83 (2023) pp. 405-412, and Emmet et al., “The PRIMARY Score: Using Intraprostatic 68Ga-PSMA PET/CT Patterns to Optimize Prostate Cancer Diagnosis,” The Journal of Nuclear Medicine 63 (2022) pp. 1644-1650, the texts of which are incorporated herein by reference in their entireties.

The PRIMARY score system takes into account uptake locations within the prostate, the peak standardized uptake value (SUV) of uptake regions of interest, the SUV value of the liver and aorta, and the shape of uptakes, and assigns a numerical grade from 1-5, where 1 indicates least intense cancer and 5 indicates most intense cancer. Staging via the PRIMARY score system offers utility across a range of indications including, for example, the staging of high-risk patients, identification and/or prediction of cancer recurrence, estimation of risk of metastases, tracking of disease progression, evaluation of suitability for radioligand therapy, and assessment of efficacy of treatment.

Currently, determination of a PRIMARY score from PSMA-PET images is not fully automated and is subject to variation. There is a need for systems and methods for more reproducible and standardized reporting of PSMA-PET staging results to support wider integration of PSMA-PET into clinical guidelines.

### SUMMARY

Presented herein are systems and methods for the automated determination of a prostate cancer staging score (e.g., a PRIMARY score) for a subject. In certain embodiments, the systems and methods employ a machine learning model (e.g., one or more convolutional neural networks, CNNs) to analyze three-dimensional (3D) images obtained via both a functional imaging modality and an anatomical imaging modality. In addition to identifying regions of PSMA binding agent uptake (hotspots), the techniques described herein are able to accurately and automatically associate specific prostate zones to each hotspot and use this information in the determination of the staging score.

Examples of the functional imaging modality include PET, SPECT (single-photon emission computerized tomography), and MRI (magnetic resonance imaging). Examples of the anatomical imaging modality includes computed tomography (CT), X-ray, and MRI. In particular embodiments, a PSMA binding agent is administered to the subject prior to obtaining the functional image (e.g., a 3D PSMA-PET image is obtained). The CT image is used to locate the prostate and/or other organs (e.g., liver and aorta) within the PSMA-PET image, and techniques described herein are used to identify uptake regions (hotspots) and, for each uptake region, identify one or more corresponding prostate zones (e.g., central, fibromuscular, peripheral, transition, and/or ureter zones). The identified and localized hotspots are then used to determine the prostate cancer staging score (e.g., the PRIMARY score) in an automated, reproducible way.

In one aspect, the invention is directed to a method for automated determination of a prostate cancer staging score (e.g., a PRIMARY score) for a subject, the method comprising: (a) receiving, by a processor of a computing device, a first image of the subject obtained using a functional imaging modality (e.g., a 3D PET, SPECT, or MRI scan) and a second image of the subject obtained using an anatomical imaging modality (e.g., a CT, X-ray, or MRI image) {e.g., receiving a combined 3D PET/CT image (e.g., a PSMA-PET/CT image) that comprises the first image and the second image}; (b) converting, by the processor, the first image into an SUV image whose intensity values correspond to standardized uptake values (SUV), thereby obtaining an SUV-converted first image; (c) localizing, by the processor, volumes of interest (VOIs) in the second image corresponding to one or more organs of the subject (e.g., a prostate, a liver, and an aorta) (e.g., using the second image to obtain one or more organ segmentation masks corresponding to one or more organs of the subject) and determining corresponding organ volumes in the SUV-converted first image (e.g., regions in the SUV-converted first image corresponding to the prostate, the liver, and/or the aorta); (d) localizing, by the processor, in the SUV-converted first image, one or more uptake regions (e.g., hotspots) corresponding to lesions (or potential lesions) in the prostate of the subject and determining, by the processor, values of an SUV uptake metric (e.g., and/or peak intensity value and/or peak intensity location) for each of the one or more uptake regions (e.g., and, optionally, determining, by the processor, whether each said uptake region is focal or diffuse), thereby determining one or more values of the SUV uptake metric; (e) for each of the one or more uptake regions, identifying, by the processor, one or more prostate zones (e.g., central, fibromuscular, peripheral, transition, and/or ureter zones) corresponding to said uptake region (e.g., by fitting a 3D prostate clinical model to the prostate segmentation mask); (f) determining, by the processor, a PSMA expression score using the SUV-converted first image (e.g., comparing a highest uptake peak within the prostate with aorta and/or liver SUV mean values); and (g) determining, by the processor, the prostate cancer staging score (e.g., the PRIMARY score) based at least on (i) the one or more values of the SUV uptake metric, (ii) the prostate zones identified for each of the one or more uptake regions (e.g., and, optionally, the determination of whether each said uptake region is focal or diffuse), and (iii) the PSMA expression score.

In certain embodiments, the first image is a three-dimensional (3D) positron emission tomography (PET) image of the subject obtained following administration to the subject of a radiopharmaceutical comprising a prostate-specific membrane antigen (PSMA) binding agent.

In certain embodiments, the PSMA binding agent comprises [¹⁸F]DCFPyL (piflufolastat F 18, e.g., PYLARIFY®,

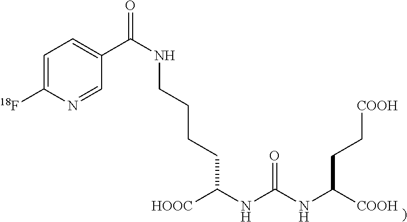

(e.g., wherein the method comprises receiving a combined 3D PSMA-PET/CT image that comprises the first image and the second image).

In certain embodiments, the PSMA binding agent comprises ⁶⁸Ga-PSMA-11 (gallium (⁶⁸Ga) gozetotide, e.g., Illucix®,

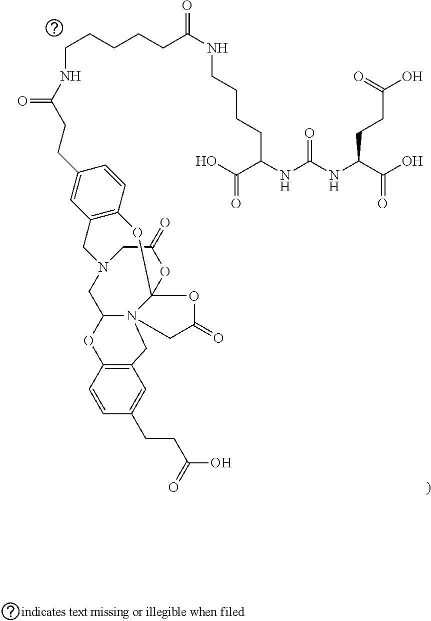

(e.g., wherein the method comprises receiving a combined 3D PSMA-PET/CT image that comprises the first image and the second image).

In certain embodiments, the SUV value determined for each of the one or more uptake regions at step (d) is a peak SUV value.

In certain embodiments, the method comprises, determining, by the processor, for each particular uptake region of the one or more uptake regions, a corresponding uptake classification label indicative of whether the particular uptake region is focal or diffuse and, at step (g) using the uptake classification labels determined for the one or more uptake regions to determine the prostate cancer staging score.

In certain embodiments, the one or more prostate zones identified for each of the one or more uptake regions are selected from a (e.g., static, finite) set of (e.g., 10 or fewer, e.g., 5 or fewer) possible prostate zones (e.g., as in an enumerated data type) (e.g., wherein the set of possible prostate zones comprises a central zone, a transition zone, and a peripheral zone; e.g., wherein the set of possible prostate zones comprises a central zone, a transition zone, and a peripheral zone, a fibromuscular zone, and an ureter zone).

In certain embodiments, identifying the one or more prostate zones (e.g., central, fibromuscular, peripheral, transition, and/or ureter zones) corresponding to each of the one or more uptake regions in the SUV-converted first image comprises, for each uptake region, (i) sorting a list of prostate zones in descending order starting from a zone in which the hotspot peak is located (e.g., location of intensity peak for the uptake region) and ending in a zone with a least number of hotspot voxels, and (ii) identify whether the uptake region extends outside the prostate.

In certain embodiments, the method comprises: localizing, by the processor, within the SUV-converted first image, a liver volume (e.g., corresponding to a liver within the subject) and/or an aorta volume (e.g., corresponding to an aorta, or portion thereof, within the subject) [e.g., the liver volume and/or aorta volume within the SUV-first image corresponding to (e.g., having been localized by mapping, to the SUV-converted first image,) a liver segmentation mask and/or an aorta segmentation mask determined from the second image); and determining, by the processor, a liver reference SUV value and/or an aorta reference SUV value based on SUV values of voxels of the SUV-converted first image within the liver volume and/or the aorta volume, respectively.

In certain embodiments, the method comprises determining the PSMA expression score based on (i) the one or more values of the SUV uptake metric and (ii) the liver reference SUV value and/or the aorta reference SUV value.

In certain embodiments, the method comprises, at step (c), localizing the VOIs in the second image using one or more machine learning module(s) [e.g., one or more convolutional neural networks (CNNs)].

In certain embodiments, the method comprises, at step (d), localizing the one or more uptake regions in the SUV-converted first image using one or more machine learning module(s) (e.g., one or more CNNs).

In another aspect, the invention is directed to a method for automated determination of a prostate cancer staging score (e.g., a PRIMARY score) for a subject, the method comprising: (a) receiving, by a processor of a computing device, data comprising a first image of the subject obtained using a functional imaging modality (e.g., a 3D PET, SPECT, or MRI scan) and a second image of the subject obtained using an anatomical imaging modality (e.g., a CT, X-ray, or MRI image) {e.g., receiving a combined 3D PET/CT image (e.g., a PSMA-PET/CT image) that comprises the first image and the second image}; (b) using the received data to localize, by the processor, one or more uptake regions (e.g., hotspots) in the first image (e.g., an SUV-converted first image) corresponding to lesions (or potential lesions) in the prostate and to identify one or more prostate zones (e.g., central, fibromuscular, peripheral, transition, and/or ureter zones) corresponding to each said uptake region (e.g., by fitting a 3D prostate clinical model to the prostate segmentation mask); and (c) determining, by the processor, the prostate cancer staging score (e.g., the PRIMARY score) based at least on the localized one or more uptake regions and their identified prostate zones.

In certain embodiments, the first image is a three-dimensional (3D) positron emission tomography (PET) image of the subject obtained following administration to the subject of a radiopharmaceutical comprising a prostate-specific membrane antigen (PSMA) binding agent.

In certain embodiments, the PSMA binding agent comprises [¹⁸F]DCFPyL (piflufolastat F 18, e.g., PYLARIFY®,

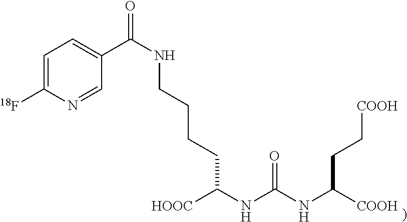

(e.g., wherein the method comprises receiving a combined 3D PSMA-PET/CT image that comprises the first image and the second image).

In certain embodiments, the PSMA binding agent comprises ⁶⁸Ga-PSMA-11 (gallium (⁶⁸Ga) gozetotide, e.g., Illucix®,

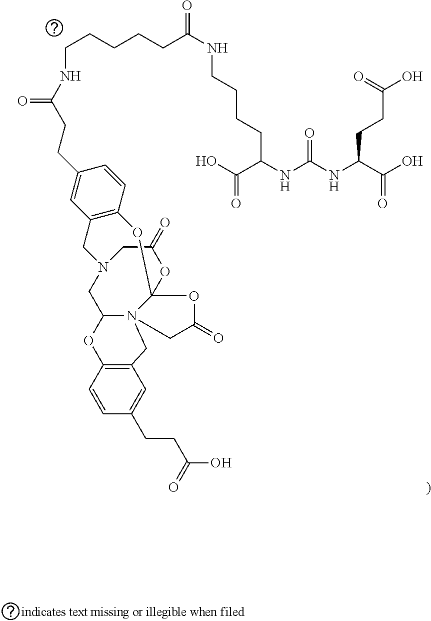

(e.g., wherein the method comprises receiving a combined 3D PSMA-PET/CT image that comprises the first image and the second image).

In certain embodiments, the method comprises, at step (b), localizing the one or more uptake regions in the first image using one or more machine learning module(s) (e.g., one or more CNNs).

In another aspect, the invention is directed to a method for automated

determination of a prostate cancer staging score for a subject, the method comprising: (a) receiving, by a processor of a computing device, a 3D functional image of the subject (e.g., a 3D PET, SPECT, or MRI scan); (b) determining, by the processor, a prostate volume within the functional image, said prostate volume identifying a region of the 3D functional image corresponding to a prostate of the subject; (c) localizing (e.g., detecting and/or segmenting), by the processor, one or more uptake regions within the 3D functional image, each determined to represent a lesion or potential lesion within the prostate of the subject or a vicinity thereof; (d) determining, by the processor, for each particular uptake region of the one or more uptake regions: (i) values of one or more uptake region intensity metrics, each corresponding to a measure of intensity within and/or characteristic of the particular uptake region [e.g., SUVmax, SUVmean, SUVpeak, etc.; e.g., a lesion index (e.g., a PSMA expression score)]; and (ii) a set of assigned prostate zones identifying, for the particular uptake region, one or more spatial zones (e.g., sub-regions) within or about the prostate of the subject that (a lesion or potential lesion represented by) the particular uptake region is associated with (e.g., within which at least a portion of a lesion or potential lesion represented by the particular uptake region is determined to be likely to be located; e.g., from which radiopharmaceutical uptake and radiation therefrom is determined to have produced the particular uptake region in the 3D functional image); and (e) determining, by the processor, the prostate cancer staging score based at least in part on (i) the values of the one or more intensity metrics and (ii) the set of assigned prostate zones determined for the one or more uptake regions.

In another aspect, the invention is directed to a system for automated determination of a prostate cancer staging score (e.g., a PRIMARY score) for a subject, the system comprising: a processor of a computing device; and memory having instructions stored thereon, wherein the memory, when executed by the processor, causes the processor to: (a) receive a first image of the subject obtained using a functional imaging modality (e.g., a 3D PET, SPECT, or MRI scan) and a second image of the subject obtained using an anatomical imaging modality (e.g., a CT, X-ray, or MRI image) {e.g., receiving a combined 3D PET/CT image (e.g., a PSMA-PET/CT image) that comprises the first image and the second image}; (b) convert the first image into an SUV image whose intensity values correspond to standardized uptake values (SUV), thereby obtaining an SUV-converted first image; (c) localize volumes of interest (VOIs) in the second image corresponding to one or more organs of the subject (e.g., a prostate, a liver, and an aorta) (e.g., using the second image to obtain one or more organ segmentation masks corresponding to one or more organs of the subject) and determining corresponding organ volumes in the SUV-converted first image (e.g., regions in the SUV-converted first image corresponding to the prostate, the liver, and/or the aorta); (d) localize, in the SUV-converted first image, one or more uptake regions (e.g., hotspots) corresponding to lesions (or potential lesions) in the prostate of the subject and determining, by the processor, values of an SUV uptake metric (e.g., and/or peak intensity value and/or peak intensity location) for each of the one or more uptake regions (e.g., and, optionally, determining, by the processor, whether each said uptake region is focal or diffuse), thereby determining one or more values of the SUV uptake metric; (e) for each of the one or more uptake regions, identify one or more prostate zones (e.g., central, fibromuscular, peripheral, transition, and/or ureter zones) corresponding to said uptake region (e.g., by fitting a 3D prostate clinical model to the prostate segmentation mask); (f) determine a PSMA expression score using the SUV-converted first image (e.g., comparing a highest uptake peak within the prostate with aorta and/or liver SUV mean values); and (g) determine the prostate cancer staging score (e.g., the PRIMARY score) based at least on (i) the one or more values of the SUV uptake metric, (ii) the prostate zones identified for each of the one or more uptake regions (e.g., and, optionally, the determination of whether each said uptake region is focal or diffuse), and (iii) the PSMA expression score.

In certain embodiments, the first image is a three-dimensional (3D) positron emission tomography (PET) image of the subject obtained following administration to the subject of a radiopharmaceutical comprising a prostate-specific membrane antigen (PSMA) binding agent.

In certain embodiments, the PSMA binding agent comprises [¹⁸F]DCFPyL (piflufolastat F 18, e.g., PYLARIFY®,

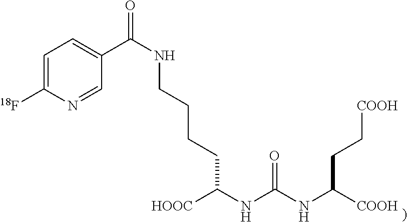

(e.g., wherein a combined 3D PSMA-PET/CT image that comprises the first image and the second image is received).

In certain embodiments, the PSMA binding agent comprises ⁶⁸Ga-PSMA-11 (gallium (⁶⁸Ga) gozetotide, e.g., Illucix®,

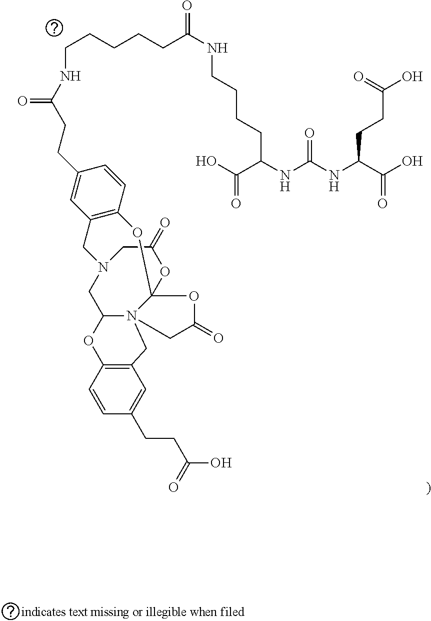

(e.g., wherein a combined 3D PSMA-PET/CT image that comprises the first image and the second image is received).

In certain embodiments, the SUV value determined for each of the one or more uptake regions at step (d) is a peak SUV value.

In certain embodiments, the instructions cause the processor to determine, for each particular uptake region of the one or more uptake regions, a corresponding uptake classification label indicative of whether the particular uptake region is focal or diffuse and, at step (g) using the uptake classification labels determined for the one or more uptake regions to determine the prostate cancer staging score.

In certain embodiments, the one or more prostate zones identified for each of the one or more uptake regions are selected from a (e.g., static, finite) set of (e.g., 10 or fewer, e.g., 5 or fewer) possible prostate zones (e.g., as in an enumerated data type) (e.g., wherein the set of possible prostate zones comprises a central zone, a transition zone, and a peripheral zone; e.g., wherein the set of possible prostate zones comprises a central zone, a transition zone, and a peripheral zone, a fibromuscular zone, and an ureter zone).

In certain embodiments, the instructions cause the processor to identify the one or more prostate zones (e.g., central, fibromuscular, peripheral, transition, and/or ureter zones) corresponding to each of the one or more uptake regions in the SUV-converted first image by, for each uptake region, (i) sorting a list of prostate zones in descending order starting from a zone in which the hotspot peak is located (e.g., location of intensity peak for the uptake region) and ending in a zone with a least number of hotspot voxels, and (ii) identify whether the uptake region extends outside the prostate.

In certain embodiments, the instructions cause the processor to: localize, within the SUV-converted first image, a liver volume (e.g., corresponding to a liver within the subject) and/or an aorta volume (e.g., corresponding to an aorta, or portion thereof, within the subject) [e.g., the liver volume and/or aorta volume within the SUV-first image corresponding to (e.g., having been localized by mapping, to the SUV-converted first image,) a liver segmentation mask and/or an aorta segmentation mask determined from the second image); and determine a liver reference SUV value and/or an aorta reference SUV value based on SUV values of voxels of the SUV-converted first image within the liver volume and/or the aorta volume, respectively.

In certain embodiments, the instructions cause the processor to determine the PSMA expression score based on (i) the one or more values of the SUV uptake metric and (ii) the liver reference SUV value and/or the aorta reference SUV value.

In certain embodiments, the instructions cause the processor to, at step (c), localize the VOIs in the second image using one or more machine learning module(s) [e.g., one or more convolutional neural networks (CNNs)].

In certain embodiments, the instructions cause the processor to, at step (d), localize the one or more uptake regions in the SUV-converted first image using one or more machine learning module(s) (e.g., one or more CNNs).

In another aspect, the invention is directed to a system for automated determination of a prostate cancer staging score (e.g., a PRIMARY score) for a subject, the system comprising: a processor of a computing device; and memory having instructions stored thereon, wherein the memory, when executed by the processor, causes the processor to: (a) receive data comprising a first image of the subject obtained using a functional imaging modality (e.g., a 3D PET, SPECT, or MRI scan) and a second image of the subject obtained using an anatomical imaging modality (e.g., a CT, X-ray, or MRI image) {e.g., receiving a combined 3D PET/CT image (e.g., a PSMA-PET/CT image) that comprises the first image and the second image}; (b) use the received data to localize one or more uptake regions (e.g., hotspots) in the first image (e.g., an SUV-converted first image) corresponding to lesions (or potential lesions) in the prostate and to identify one or more prostate zones (e.g., central, fibromuscular, peripheral, transition, and/or ureter zones) corresponding to each said uptake region (e.g., by fitting a 3D prostate clinical model to the prostate segmentation mask); and (c) determine the prostate cancer staging score (e.g., the PRIMARY score) based at least on the localized one or more uptake regions and their identified prostate zones.

In certain embodiments, the first image is a three-dimensional (3D) positron emission tomography (PET) image of the subject obtained following administration to the subject of a radiopharmaceutical comprising a prostate-specific membrane antigen (PSMA) binding agent.

In certain embodiments, the PSMA binding agent comprises [¹⁸F]DCFPyL (piflufolastat F 18, e.g., PYLARIFY®,

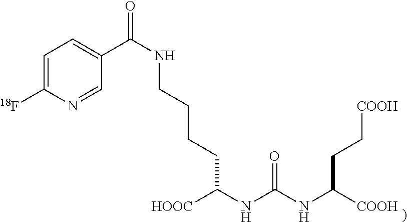

(e.g., wherein the method comprises receiving a combined 3D PSMA-PET/CT image that comprises the first image and the second image).

In certain embodiments, the PSMA binding agent comprises ⁶⁸Ga-PSMA-11 (gallium (⁶⁸Ga) gozetotide, e.g., Illucix®,

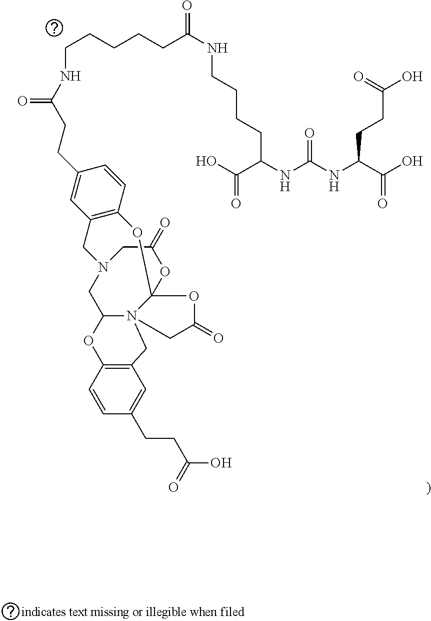

(e.g., wherein the method comprises receiving a combined 3D PSMA-PET/CT image that comprises the first image and the second image).

In certain embodiments, the instructions cause the processor to, at step (b), localize the one or more uptake regions in the first image using one or more machine learning module(s) (e.g., one or more CNNs).

In another aspect, the invention is directed to a system for automated determination of a prostate cancer staging score for a subject, the system comprising: a processor of a computing device; and memory having instructions stored thereon, wherein the memory, when executed by the processor, causes the processor to: (a) receive a 3D functional image of the subject (e.g., a 3D PET, SPECT, or MRI scan); (b) determine a prostate volume within the functional image, said prostate volume identifying a region of the 3D functional image corresponding to a prostate of the subject; (c) localize (e.g., detect and/or segment) one or more uptake regions within the 3D functional image, each determined to represent a lesion or potential lesion within the prostate of the subject or a vicinity thereof; (d) determine, for each particular uptake region of the one or more uptake regions: (i) values of one or more uptake region intensity metrics, each corresponding to a measure of intensity within and/or characteristic of the particular uptake region [e.g., SUVmax, SUVmean, SUVpeak, etc.; e.g., a lesion index (e.g., a PSMA expression score)]; and (ii) a set of assigned prostate zones identifying, for the particular uptake region, one or more spatial zones (e.g., sub-regions) within or about the prostate of the subject that (a lesion or potential lesion represented by) the particular uptake region is associated with (e.g., within which at least a portion of a lesion or potential lesion represented by the particular uptake region is determined to be likely to be located; e.g., from which radiopharmaceutical uptake and radiation therefrom is determined to have produced the particular uptake region in the 3D functional image); and (e) determine the prostate cancer staging score based at least in part on (i) the values of the one or more intensity metrics and (ii) the set of assigned prostate zones determined for the one or more uptake regions.

In certain embodiments, elements described with respect to one aspect of the invention are implemented in another aspect of the invention described herein.

### BRIEF DESCRIPTION OF THE DRAWING

The foregoing and other objects, aspects, features, and advantages of the present disclosure will become more apparent and better understood by referring to the following description taken in conjunction with the accompanying drawings, in which:

FIG. 1 is an image cube (e.g., a set of PET images) showing a prostate, cropped from a full-size PET image, and indicating a number of slices in every dimension, according to an illustrative embodiment.

FIG. 2 is a set of three images showing corresponding slices of a CT image, a PET image, and a PET/CT fusion, obtained from a 3D PET/CT scan, according to an illustrative embodiment.

FIG. 3 is a diagram illustrating an example process for segmenting an anatomical image and identifying anatomical boundaries in a co-aligned functional image, according to an illustrative embodiment.

FIG. 4 is a diagram illustrating an example process for segmenting and classifying hotspots, according to an illustrative embodiment.

FIG. 5A is a schematic showing an approach for computing lesion index values, according to an illustrative embodiment.

FIG. 5B is a schematic showing another approach for computing lesion index values, according to an illustrative embodiment.

FIG. 6 is a block flow diagram of an example process for automated determination of a prostate cancer staging score, according to an illustrative embodiment.

FIG. 7 is a block diagram of an exemplary cloud computing environment, used in certain embodiments.

FIG. 8 is a block diagram of an example computing device and an example mobile computing device used in certain embodiments.

FIG. 9 is a block flow diagram of an illustrative method for automated determination of a PRIMARY score for prostate cancer staging, according to an illustrative embodiment.

FIG. 10A is an example image of prostate-located uptake regions (hotspots) that is classified as “diffuse”, according to an illustrative embodiment.

FIG. 10B is an example image of prostate-located uptake regions (hotspots) that is classified as “focal”, according to an illustrative embodiment.

FIG. 11 is a graphical representation of a three-dimensional (3D) clinical prostate model used to identify one or more zones within the prostate in which each uptake region (hotspot) is located, according to an illustrative embodiment.

FIG. 12 is a graphical representation of the clinical prostate model fit to a prostate segmentation mask in the CT image, according to an illustrative embodiment.

FIG. 13 is a compilation of images depicting an example automated computation of a PRIMARY score, according to an illustrative embodiment.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### CERTAIN DEFINITIONS

In order for the present disclosure to be more readily understood, certain terms are first defined below. Additional definitions for the following terms and other terms are set forth throughout the specification.

A, an: The articles “a” and “an” are used herein to refer to one or to more than one (i.e., at least one) of the grammatical object of the article. By way of example, “an element” means one element or more than one element. Thus, in this specification and the appended claims, the singular forms “a,” “an,” and “the” include plural references unless the context clearly dictates otherwise. Thus, for example, reference to a pharmaceutical composition comprising “an agent” includes reference to two or more agents.

About, approximately: As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

First, second, etc.: It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed or that the first element must precede the second element in some manner. In addition, unless stated otherwise, a set of elements may comprise one or more elements.

Administering: As used herein, “administering” an agent means introducing a substance (e.g., an imaging agent) into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments.

3D, three-dimensional: As used herein, “3D” or “three-dimensional” with reference to an “image” means conveying information about three dimensions. A 3D image may be rendered as a dataset in three dimensions and/or may be displayed as a set of two-dimensional representations, or as a three-dimensional representation. In certain embodiments, a 3D image is represented as voxel (e.g., volumetric pixel) data.

Image: As used herein, an “image”—for example, a three-dimensional (3D) image of subject, includes any visual representation, such as a photo, a video frame, streaming video, as well as any electronic, digital or mathematical analogue of a photo (e.g., a digital image), video frame, or streaming video, displayed or stored in memory (e.g., a digital image may, but need not be displayed for visual inspection). Any apparatus described herein, in certain embodiments, includes a display for displaying an image or any other result produced by the processor. Any method described herein, in certain embodiments, includes a step of displaying an image or any other result produced via the method. In certain embodiments, an image is a 3D image, conveying information that varies with position within a 3D volume. Such images may, for example, be represented digitally as a 3D matrix (e.g., a N×M×L matrix) with each voxel of a 3D image represented by an element of a 3D matrix. Other representations are also contemplated and included, for example, a 3D matrix may be reshaped as a vector (e.g., a 1×K size vector, where K is a total number of voxels) by stitching each row or column end to end. Examples of images include, for example, medical images, such as bone-scan images (also referred to as scintigraphy images), computed tomography (CT) images, magnetic resonance images (MRIs), optical images (e.g., bright-field microscopy images, fluorescence images, reflection or transmission images, etc.), positron emission tomography (PET) images, single-photon emission tomography (SPECT) images, ultrasound images, x-ray images, and the like. In certain embodiments, a medical image is or comprises a nuclear medicine image, produced from radiation emitted from within a subject being imaged. In certain embodiments, a medical image is or comprises an anatomical image (e.g., a 3D anatomical image) conveying information regarding location and extent of anatomical structures such as internal organs, bones, soft-tissue, and blood vessels, within a subject. Examples of anatomical images include, without limitation, x-ray images, CT images, MRIs, and ultrasound images. In certain embodiments, a medical image is or comprises a functional image (e.g., a 3D functional image) conveying information relating to physiological activities within specific organs and/or tissue, such as metabolism, blood flow, regional chemical composition, absorption, etc. Examples of functional images include, without limitation, nuclear medicine images, such as PET images, SPECT images, as well as other functional imaging modalities, such as functional MRI (fMRI), which measures small changes in blood flow for use in assessing brain activity.

Radionuclide: As used herein, “radionuclide” refers to a moiety comprising a radioactive isotope of at least one element. Exemplary suitable radionuclides include but are not limited to those described herein. In some embodiments, a radionuclide is one used in positron emission tomography (PET). In some embodiments, a radionuclide is one used in single-photon emission computed tomography (SPECT). In some embodiments, a non-limiting list of radionuclides includes ^{99m}Tc, ¹¹¹In, ⁶⁴Cu, ⁶⁷Ga, ⁶⁸Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹²⁶I, ¹³¹I, ¹¹C, ¹³N, ¹⁵O, ¹⁸F, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, ⁸²Rb, ⁷⁵Br, ⁷⁶Br, ⁷⁷Br, ⁸⁰Br, ^{80m}Br, ⁸²Br, ⁸³Br, ²¹¹At and ¹⁹²Ir.

Radiopharmaceutical: As used herein, the term “radiopharmaceutical” refers to a compound comprising a radionuclide. In certain embodiments, radiopharmaceuticals are used for diagnostic and/or therapeutic purposes. In certain embodiments, radiopharmaceuticals include small molecules that are labeled with one or more radionuclide(s), antibodies that are labeled with one or more radionuclide(s), and antigen-binding portions of antibodies that are labeled with one or more radionuclide(s).

Machine learning module: Certain embodiments described herein make use of (e.g., include) software instructions that include one or more machine learning module(s), also referred to herein as artificial intelligence software. As used herein, the term “machine learning module” refers to a computer implemented process (e.g., function) that implements one or more specific machine learning algorithms in order to determine, for a given input (such as an image (e.g., a 2D image; e.g., a 3D image), dataset, and the like) one or more output values. For example, a machine learning module may receive as input a 3D image of a subject (e.g., a CT image; e.g., an MRI), and for each voxel of the image, determine a value that represents a likelihood that the voxel lies within a region of the 3D image that corresponds to a representation of a particular organ or tissue of the subject. In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions of a CNN module may be carried out via specialized hardware (e.g., via an application specific integrated circuit (ASIC)).

Map: As used herein, the term “map” is understood to mean a visual display, or any data representation that may be interpreted for visual display, which contains spatially-correlated information. For example, a three-dimensional map of a given volume may include a dataset of values of a given quantity that varies in three spatial dimensions throughout the volume. A three-dimensional map may be displayed in two-dimensions (e.g., on a two-dimensional screen, or on a two-dimensional printout).

Segmentation map: As used herein, the term “segmentation map” refers to a computer representation that identifies one or more 2D or 3D regions determined by segmenting an image. In certain embodiments, a segmentation map distinguishably identifies multiple different (e.g., segmented) regions, allowing them to be individually and distinguishably accessed and operated upon and/or used for operating on, for example, one or more images.

Subject: As used herein, a “subject” means a human or other mammal (e.g., rodent (mouse, rat, hamster), pig, cat, dog, horse, primate, rabbit, and the like). The term “subject” is used herein interchangeably with the term “patient”.

Tissue: As used herein, the term “tissue” refers to bone (osseous tissue) as well as soft-tissue.

Whole body: As used herein, the terms “full body” and “whole body” used (interchangeably) in the context of segmentation and other manners of identification of regions within an image of a subject refer to approaches that evaluate a majority (e.g., greater than 50%) of a graphical representation of a subject's body in a 3D anatomical image to identify target tissue regions of interest. In certain embodiments, full body and whole-body segmentation refers to identification of target tissue regions within at least an entire torso of a subject. In certain embodiments, portions of limbs are also included, along with a head of the subject.

### DETAILED DESCRIPTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader-the presence and/or placement of a header is not intended to limit the scope of the subject matter described herein.

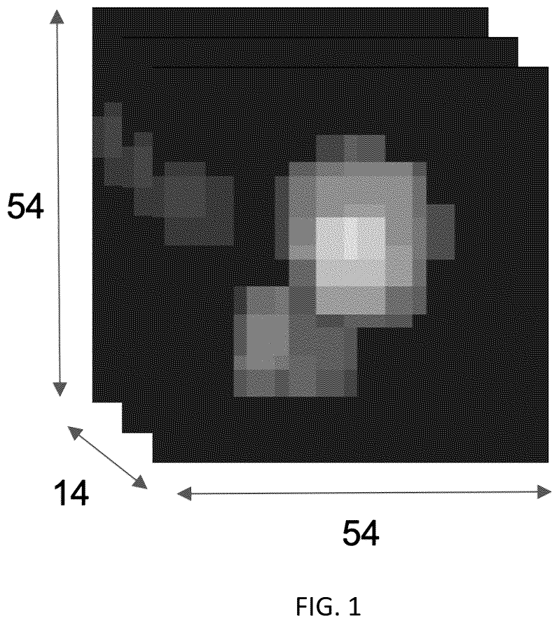

As described in further detail herein systems and methods of the present disclosure provide technologies for automated determination of prostate cancer staging scores, whereby intensity patterns of uptake regions identified within and/or about a prostate volume in a 3D functional image are used to assign a patient's cancer a grade indicative of its malignancy. For example, as shown in FIG. 1, intensity patterns in 3D functional images, such as PET images, reflect radiopharmaceutical uptake within a patient, desirably concentrated within cancerous tissue, and can display a variety of spatial patterns that can be challenging to interpret and, moreover, difficult to compare with, for example, other patients (e.g., in a cohort), reference images (e.g., to inform treatment decisions), or images taken at earlier time points (e.g., to assess disease progression and/or treatment efficacy) in an objective fashion. Accordingly, by converting complex spatial intensity patterns to a numerical grade on a scale, approaches described herein facilitate evaluation of patients for prostate cancer and can help to inform treatment decisions.

Moreover, although grading schemes, such as the PRIMARY scoring technique described in Emmet et al., “The PRIMARY Score: Using Intraprostatic 68Ga-PSMA PET/CT Patterns to Optimize Prostate Cancer Diagnosis,” The Journal of Nuclear Medicine 63 (2022) pp. 1644-1650, have been proposed for use by medical professionals, human reader assessments and interpretation of images, particularly those involving complex spatial intensity patterns, are time consuming and subjective—resulting in inefficiencies and inter-reader variability that limits their practical value. Accordingly, by leveraging and combining various approaches for automatically identifying anatomical regions and lesions in medical images, and quantifying their severity/uptake, technologies of the present disclosure provide automated, efficient, and robust techniques for scoring prostate cancer images in a manner can provide for improved accuracy and consistency in assessment of patients' disease.

### A. Nuclear Medicine Images

In certain embodiments, technologies of the present disclosure analyze nuclear medicine images. Nuclear medicine images may be obtained using a nuclear medicine imaging modality such as bone scan imaging (also referred to as scintigraphy), Positron Emission Tomography (PET) imaging, and Single-Photon Emission Tomography (SPECT) imaging.

In certain embodiments, nuclear medicine images are obtained using imaging agents comprising radiopharmaceuticals. Nuclear medicine images may be obtained following administration of a radiopharmaceutical to a patient (e.g., a human subject), and provide information regarding the distribution of the radiopharmaceutical within the patient.

Nuclear medicine imaging techniques detect radiation emitted from the radionuclides of radiopharmaceuticals to form an image. The distribution of a particular radiopharmaceutical within a patient may be influenced and/or dictated by biological mechanisms such as blood flow or perfusion, as well as by specific enzymatic or receptor binding interactions. Different radiopharmaceuticals may be designed to take advantage of different biological mechanisms and/or particular specific enzymatic or receptor binding interactions and thus, when administered to a patient, selectively concentrate within particular types of tissue and/or regions within the patient. Greater amounts of radiation are emitted from regions within the patient that have higher concentrations of radiopharmaceutical than other regions, such that these regions appear brighter in nuclear medicine images. Accordingly, intensity variations within a nuclear medicine image can be used to map the distribution of radiopharmaceutical within the patient. This mapped distribution of radiopharmaceutical within the patient can be used to, for example, infer the presence of cancerous tissue within various regions of the patient's body. In certain embodiments, intensities of voxels of a nuclear medicine image, for example a PET image, represent standardized uptake values (SUVs) (e.g., having been calibrated for injected radiopharmaceutical dose and/or patient weight, for example as shown in Section H.i., below).

For example, upon administration to a patient, technetium 99m methylenediphosphonate (^{99m}Tc MDP) selectively accumulates within the skeletal region of the patient, in particular at sites with abnormal osteogenesis associated with malignant bone lesions. The selective concentration of radiopharmaceutical at these sites produces identifiable hotspots-localized regions of high intensity-in nuclear medicine images. Accordingly, presence of malignant bone lesions associated with metastatic prostate cancer can be inferred by identifying such hotspots within a whole-body scan of the patient. In certain embodiments, analyzing intensity variations in whole-body scans obtained following administration of ^{99m}Tc MDP to a patient, such as by detecting and evaluating features of hotspots, can be used to compute, risk indices that correlate with patient overall survival and other prognostic metrics indicative of disease state, progression, treatment efficacy, and the like. In certain embodiments, other radiopharmaceuticals can also be used in a similar fashion to ^{99m}Tc MDP.

In certain embodiments, the particular radiopharmaceutical used depends on the particular nuclear medicine imaging modality used. For example, 18F sodium fluoride (NaF) also accumulates in bone lesions, similar to ^{99m}Tc MDP, but can be used with PET imaging. In certain embodiments, PET imaging may also utilize a radioactive form of the vitamin choline, which is readily absorbed by prostate cancer cells.

In certain embodiments, radiopharmaceuticals that selectively bind to particular proteins or receptors of interest—particularly those whose expression is increased in cancerous tissue may be used. Such proteins or receptors of interest include, but are not limited to tumor antigens, such as CEA, which is expressed in colorectal carcinomas, Her2/neu, which is expressed in multiple cancers, BRCA 1 and BRCA 2, expressed in breast and ovarian cancers; and TRP-1 and -2, expressed in melanoma.

For example, human prostate-specific membrane antigen (PSMA) is upregulated in prostate cancer, including metastatic disease. PSMA is expressed by virtually all prostate cancers and its expression is further increased in poorly differentiated, metastatic and hormone refractory carcinomas. Accordingly, radiopharmaceuticals that comprise PSMA binding agents (e.g., compounds that a high affinity to PSMA) labelled with one or more radionuclide(s) can be used to obtain nuclear medicine images of a patient from which the presence and/or state of prostate cancer within a variety of regions (e.g., including, but not limited to skeletal regions) of the patient can be assessed. In certain embodiments, nuclear medicine images obtained using PSMA binding agents are used to identify the presence of cancerous tissue within the prostate, when the disease is in a localized state. In certain embodiments, nuclear medicine images obtained using radiopharmaceuticals comprising PSMA binding agents are used to identify the presence of cancerous tissue within a variety of regions that include not only the prostate, but also other organs and tissue regions such as lungs, lymph nodes, and bones, as is relevant when the disease is metastatic.

In particular, upon administration to a patient, radionuclide labelled PSMA binding agents selectively accumulate within cancerous tissue, based on their affinity to PSMA. In a similar manner to that described above with regard to ^{99m}Tc MDP, the selective concentration of radionuclide labelled PSMA binding agents at particular sites within the patient produces detectable hotspots in nuclear medicine images. As PSMA binding agents concentrate within a variety of cancerous tissues and regions of the body expressing PSMA, localized cancer within a prostate of the patient and/or metastatic cancer in various regions of the patient's body can be detected, and evaluated. Various metrics that are indicative of and/or quantify severity (e.g., likely malignancy) of individual lesions, overall disease burden and risk for a patient, and the like, can be computed based on automated analysis of intensity variations in nuclear medicine images obtained following administration of a PSMA binding agent radiopharmaceutical to a patient. These disease burden and/or risk metrics may be used to stage disease and make assessments regarding patient overall survival and other prognostic metrics indicative of disease state, progression, treatment efficacy,

A variety of radionuclide labelled PSMA binding agents may be used as radiopharmaceutical imaging agents for nuclear medicine imaging to detect and evaluate prostate cancer. In certain embodiments, the particular radionuclide labelled PSMA binding agent that is used depends on factors such as the particular imaging modality (e.g., PET; e.g., SPECT) and the particular regions (e.g., organs) of the patient to be imaged. For example, certain radionuclide labelled PSMA binding agents are suited for PET imaging, while others are suited for SPECT imaging. For example, certain radionuclide labelled PSMA binding agents facilitate imaging a prostate of the patient, and are used primarily when the disease is localized, while others facilitate imaging organs and regions throughout the patient's body, and are useful for evaluating metastatic prostate cancer.

Several exemplary PSMA binding agents and radionuclide labelled versions thereof are described in further detail in Section H herein, as well as in U.S. Pat. Nos. 8,778,305, 8,211,401, and 8,962,799, and in U.S. Patent Publication No. US 2021/0032206 A1, the content of each of which are incorporated herein by reference in their entireties.

### B. Image Segmentation in Nuclear Medicine Imaging

Nuclear medicine images are functional images. Functional images convey information relating to physiological activities within specific organs and/or tissue, such as metabolism, blood flow, regional chemical composition, and/or absorption. In certain embodiments, nuclear medicine images are acquired and/or analyzed in combination with anatomical images, such as computed tomography (CT) images. Anatomical images provide information regarding location and extent of anatomical structures such as internal organs, bones, soft-tissue, and blood vessels, within a subject. Examples of anatomical images include, without limitation, x-ray images, CT images, magnetic resonance images, and ultrasound images.

Accordingly, in certain embodiments, anatomical images can be analyzed together with nuclear medicine images in order to provide anatomical context for the functional information that they (nuclear medicine images) convey. For example, while nuclear medicine images, such as PET and SPECT convey a three-dimensional distribution of radiopharmaceutical within a subject, adding anatomical context from an anatomical imaging modality, such as CT imaging, allows one to determine the particular organs, soft-tissue regions, bones, etc. that radiopharmaceutical has accumulated in.

For example, a functional image may be aligned with an anatomical image so that locations within each image that correspond to a same physical location—and therefore correspond to each other—can be identified. For example, coordinates and/or pixels/voxels within a functional image and an anatomical image may be defined with respect to a common coordinate system, or a mapping (i.e., a functional relationship) between voxels within the anatomical image and voxels within the functional image established. In this manner, one or more voxels within an anatomical image and one or more voxels within a functional image that represent a same physical location or volume can be identified as corresponding to each other.

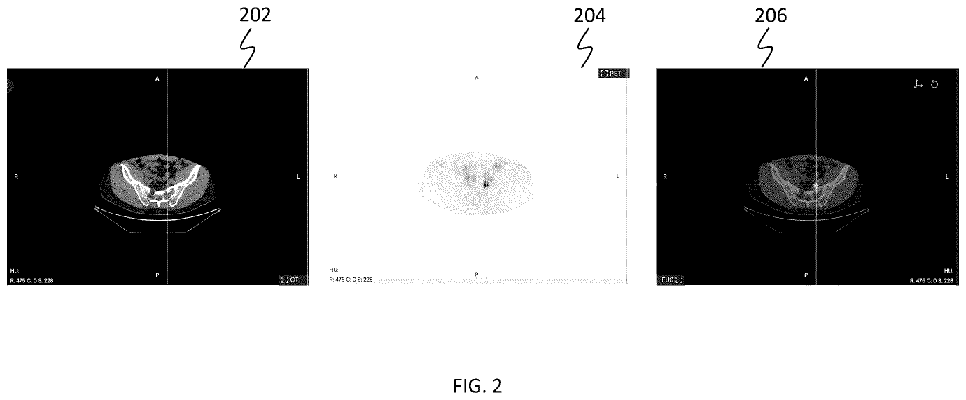

For example, FIG. 2 shows axial slices of a 3D CT image 202 and a 3D PET image 204, along with a fused image 206 in which the slice of the 3D CT image is displayed in grayscale and with the PET image is displayed as a semitransparent overlay. By virtue of the alignment between the CT and PET images, a location of a hotspot within the PET image, indicative of accumulated radiopharmaceutical and, accordingly a potential lesion, can be identified in the corresponding CT image, and viewed in anatomical context, for example, within a particular location in the pelvic region (e.g., within a prostate).

In certain embodiments, the aligned pair are a composite image, such as a PET/CT or SPECT/CT. In certain embodiments, an anatomical image (e.g., a 3D anatomical image, such as a CT image) and a functional image (e.g., a 3D functional image, such as a PET or SPECT image) are acquired using separate anatomical and functional imaging modalities, respectively. In certain embodiments, an anatomical image (e.g., a 3D anatomical image, such as a CT image) and a functional image (e.g., a 3D functional image, such as a PET or SPECT image) are acquired using a single multimodality imaging system. A functional image and an anatomical image may, for example, be acquired via two scans using a single multimodal imaging system-for example first performing a CT scan and then, second, performing a PET scan—during which a subject remains in a substantially fixed position.

In certain embodiments, 3D boundaries of particular tissue regions of interest can be accurately identified by analyzing 3D anatomical images. For example, automated segmentation of 3D anatomical images can be performed to segment 3D boundaries of regions such as particular organs, organ sub-regions and soft-tissue regions, as well as bone. In certain embodiments, organs such as a prostate, urinary bladder, liver, aorta (e.g., portions of an aorta, such as a thoracic aorta), a parotid gland, etc., are segmented. In certain embodiments, one or more particular bones are segmented. In certain embodiments, an overall skeleton is segmented.

In certain embodiments, automated segmentation of 3D anatomical images may be performed using one or more machine learning modules that are trained to receive a 3D anatomical image and/or a portion thereof, as input, and segment one or more particular regions of interest, producing a 3D segmentation map as output. For example as described in PCT publication WO/2020/144134, entitled “Systems and Methods for Platform Agnostic Whole Body Segmentation,” and published Jul. 16, 2020, the contents of which are incorporated herein by reference in their entirety, multiple machine learning modules implementing convolutional neural networks (CNNs) may be used to segment 3D anatomical images, such as CT images, of a whole body of a subject and thereby create a 3D segmentation map that identifies multiple target tissue regions across a subject's body.

In certain embodiments, for example to segment certain organs where functional images are believed to provide additional useful information that facilitate segmentation, a machine learning module may receive both an anatomical image and a functional image as input, for example as two different channels of input (e.g., analogous to multiple color channels in a color, RGB, image) and use these two inputs to determine an anatomical segmentation. This, multi-channel, approach is described in further detail in U.S. Patent Publication No. US 2021/0334974 A1, entitled “Systems and Methods for Deep-Learning-Based Segmentation of Composite Images,” and published Oct. 28, 2021, the contents of which is hereby incorporated by reference in its entirety.

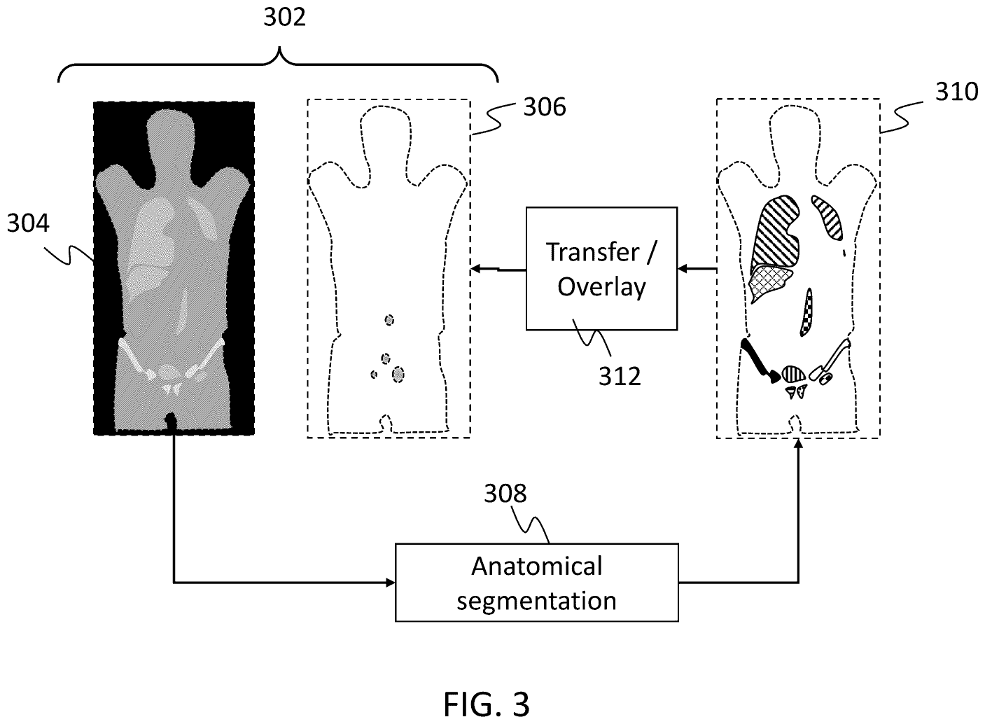

In certain embodiments, as illustrated FIG. 3, an anatomical image 304 (e.g., a 3D anatomical image, such as a CT image) and a functional image 306 (e.g., a 3D functional image, such as a PET or SPECT image) may be aligned with (e.g., co-registered to) each other, for example as in a composite image 302 such as a PET/CT image. Anatomical image 304 may be segmented 308 to create a segmentation map 310 (e.g., a 3D segmentation map) that distinguishably identifies one or more tissue regions and/or sub-regions of interest, such as one or more particular organs and/or bones. Segmentation map 310, having been created from anatomical image 304 is aligned with anatomical image 304, which, in turn, is aligned with functional image 306. Accordingly, boundaries of particular regions (e.g., segmentation masks), such as particular organs and/or bones, identified via segmentation map 210 can be transferred to and/or overlaid 312 upon functional image 306 to identify volumes within functional image 306 for purposes of classifying hotspots, and determining useful indices that serve as measures and/or predictions of cancer status, progression, and response to treatment. Segmentation maps and masks may also be displayed, for example as a graphical representation overlaid on a medical image to guide physicians and other medical practitioners.

### C. Lesion Detection and Characterization

In certain embodiments, approaches described herein include techniques for detecting and characterizing lesions within a subject via (e.g., automated) analysis of medical images, such as nuclear medicine images. Regions of interest (ROIs) in medical images that represent potential lesions may be identified based on, for example, differences in intensity values relative to surroundings, or other characteristic features (e.g., abnormal shapes, texture, spatial frequencies, etc., depending on particular imaging modality). That is, in certain embodiments, ROIs representing potential lesion (also referred to as “suspect regions”) may appear in medical images as spatial regions having intensity values and/or patters deviating from an established baseline pattern, e.g., background intensities. In certain embodiments, suspect ROIs are uptake regions that are indicative of anomalous, e.g., increased, uptake of an imaging agent within a particular localized region within a patient. In particular, as described herein, in certain embodiments, uptake regions may be hotspots. In certain embodiments, hotspots are localized (e.g., contiguous) regions of high intensity, relative to their surroundings, within images, such as 3D functional images and may be indicative of a potential cancerous lesion present within a subject.

A variety of approaches may be used for detecting, segmenting, and classifying uptake regions such as hotspots. In certain embodiments, hotspots are detected and segmented using analytical methods, such as filtering techniques including, but not limited to, a difference of Gaussians (DoG) filter and a Laplacian of Gaussians (LoG) filter. In certain embodiments, hotspots are segmented using a machine learning module that receives, as input, a 3D functional image, such as a PET image, and generates, as output a hotspot segmentation map (a “hotspot map”) that differentiates boundaries of identified hotspots from background. In certain embodiments, each segmented hotspot within a hotspot map is individually identifiable (e.g., individually labelled). In certain embodiments, a machine learning module used for segmenting hotspots may take as input, in addition to a 3D functional image, one or both of a 3D anatomical image (e.g., a CT image) and a 3D anatomical segmentation map. The 3D anatomical segmentation map may be generated via automated segmentation (e.g., as described herein) of the 3D anatomical image.

In certain embodiments, segmented hotspots may be classified according to an anatomical region in which they are located. For example, in certain embodiments, locations of individual segmented hotspots within a hotspot map (representing and identifying segmented hotspots) may be compared with 3D boundaries of segmented tissue regions, such as various organs and bones, within a 3D anatomical segmentation map and labeled according to their location, e.g., based on proximity to and/or overlap with particular organs. In certain embodiments, a machine learning module may be used to classify hotspots. For example, in certain embodiments, a machine learning module may generate, as output, a hotspot map in which segmented hotspots are not only individually labeled and identifiable (e.g., distinguishable from each other), but are also labeled, for example, as corresponding to one of a bone, lymph, or prostate lesion. In certain embodiments, one or more machine learning modules may be combined with each other, as well as with analytical segmentation (e.g., thresholding) techniques to perform various tasks in parallel and in sequence to create a final labeled hotspot map.

Various approaches for performing detailed segmentation of 3D anatomical images and identification of hotspots representing lesions in 3D functional images, which may be used with various approaches described herein, are described in PCT publication WO/2020/144134, entitled “Systems and Methods for Platform Agnostic Whole Body Segmentation,” and published Jul. 16, 2020, U.S. Patent Publication No. US 2021/0334974 A1, entitled “Systems and Methods for Deep-Learning-Based Segmentation of Composite Images,” and published Oct. 28, 2021, and PCT publication WO/2022/008374, entitled “Systems and Methods for Artificial Intelligence-Based Image Analysis for Detection and Characterization of Lesions,” and published Jan. 13, 2022, the contents of each of which is incorporated herein in its entirety.

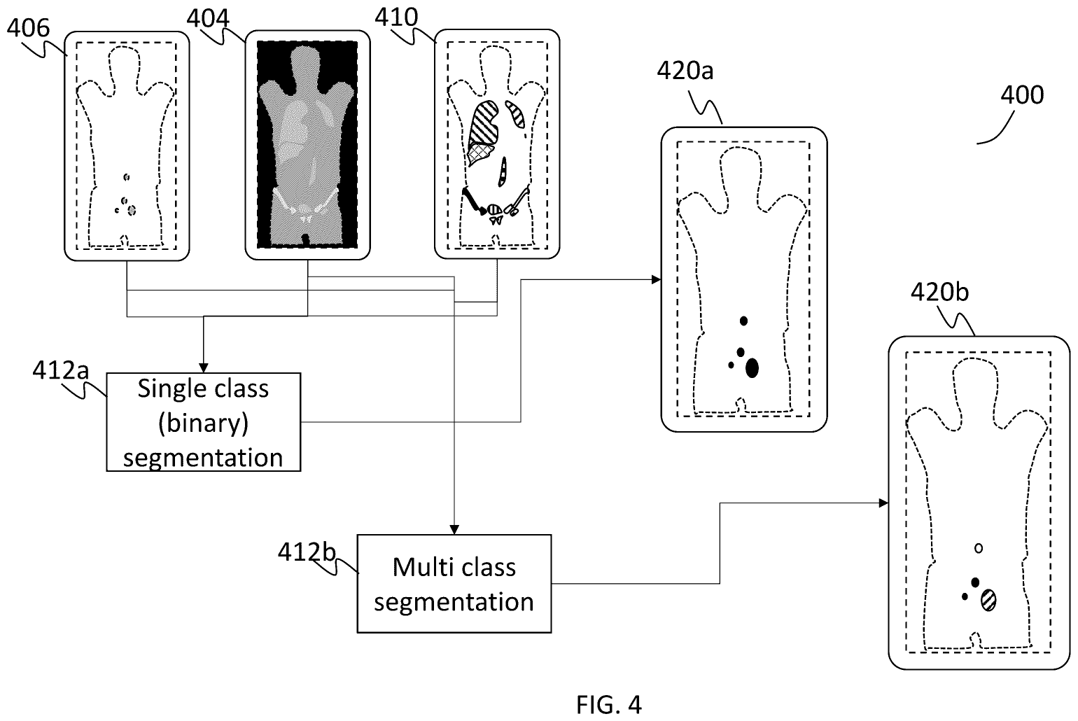

FIG. 4 shows an example process 400 for segmenting and classifying hotspots, based on an example approach described in further detail in PCT publication WO/2022/008374, entitled “Systems and Methods for Artificial Intelligence-Based Image Analysis for Detection and Characterization of Lesions,” and published Jan. 13, 2022. The approach illustrated in FIG. 3 uses two machine learning modules, each of which receives, as input, 3D functional image 406, 3D anatomical image 404, and 3D anatomical segmentation map 410. Machine learning module 412a is a binary classifier that generates a single-class hotspot map 420a, by labeling voxels as hotspot or background (not a hotspot). Machine learning module 412b performs multi-class segmentation, and generates multi-class hotspot map 420b, in which hotspots are both segmented and labeled as one of three classes—prostate, lymph, or bone. Among other things, classifying hotspots in this manner—via a machine learning module 412b (e.g., as opposed to directly comparing hotspot locations with segmented boundaries from segmentation map 410)—obviates a need to segment certain regions. For example, in certain embodiments, machine learning module 312b may classify hotspots as belonging to prostate, lymph, or bone, without a prostate region having be identified and segmented from 3D anatomical image 404 (e.g., in certain embodiments, 3D anatomical segmentation map 410 does not comprise a prostate region). In certain embodiments, hotspot maps 420a and 420b are merged, for example by transferring labels from multi-class hotspot map 420b to the hotspot segmentations identified in single-class hotspot map 420a (e.g., based on overlap). Without wishing to be bound to any particular theory, it is believed that this approach combines improved segmentation and detection of hotspots from single class machine learning module 412a with classification results from multi-class machine learning module 412b. In certain embodiments, hotspot regions identified via this final, merged, hotspot map are further refined, using an analytical technique such as an adaptive thresholding technique described in PCT publication WO/2022/008374, entitled “Systems and Methods for Artificial Intelligence-Based Image Analysis for Detection and Characterization of Lesions,” and published Jan. 13, 2022.

In certain embodiments, once detected and segmented, hotspots may be identified and assigned labels according to a particular anatomical (e.g., tissue) region in which they are located and/or a particular lesion sub-type that they are likely to represent. For example, in certain embodiments, hotspots may be assigned an anatomical location that identifies them as representing locations with a one of a set of tissue regions, such as the listed in Table 1, below. In certain embodiments, a list of tissue regions may include those in Table 1 as well as a gluteus maximus (e.g., left and right) and a gallbladder. In certain embodiments, hotspots are assigned to and/or labeled as belonging to a particular tissue region based on a machine learning classification and/or via comparison of their 3D hotspot volume's location and/or overlap with various tissue volumes identified via masks in an anatomical segmentation map. In certain embodiments, a prostate is not segmented. For example, as described above, in certain embodiments, machine learning module 312b may classify hotspots as belonging to prostate, lymph, or bone, without a prostate region having be identified and segmented from 3D anatomical image 304. In certain embodiments, regions such as a head and/or one or more arm(s) of a patient, may be segmented, for example as shown in FIG. 4A. In certain embodiments, one or more arm(s) comprises upper arm(s) and/or at least a portion of one or both humerus/humeri. In certain embodiments, a head comprises a skull, a mandible, a brain, a parotid gland, and cervical curve, and a cervical vertebra. In certain embodiments, segmentation processes of the present disclosure may utilize a cutoff rule to determine whether to segment a particular region based on an extent of a subject that is imaged, and/or is graphically represented in a medical image. For example, in certain embodiments, if a medical image does not cover (e.g., certain percentage of a region of interest, reach set anatomical landmarks), segmentation of a particular region (e.g., a skull, a parotid gland) is not performed, or may be performed, but discarded or flagged (e.g., as low confidence/potentially erroneous).

In certain embodiments, technologies of the present disclosure provide users with tools that include segmentation procedures that utilize particular, processes for detecting and/or segmenting hotspots that are tailored to different regions (e.g., organs). For example, a user may, seek to locate hotspots in an organ with high physiological uptake, such as a liver that an approach used for (detecting and/or segmenting hotspots within) other organs may struggle with. Accordingly, a tailored approach for detecting and/or segmenting hotspots in high uptake organs may be used.

In certain embodiments, a hotspot segmentation procedure for organ with high physiological uptake may include one or both of: (1) detecting hotspots by applying a model (e.g., statistical, machine-learning) to determine and remove (e.g., filter out) intensities corresponding to normal, background, uptake within the organ; and (2) segmenting hotspots by applying a model (e.g., statistical, machine-learning) to identify and delineate 3D hotspot volume boundaries using the organ boundaries (e.g., which may be identified via one or more segmentation masks determined via approaches described herein, for example, in section B above) and/or determined local background intensity value associated with and representing normal background uptake within the organ (e.g., by detecting abnormally high intensity peaks).

FIG. 4B shows an example process 410 for detecting hotspots in a high uptake organ according to various embodiments described herein. A 3D functional image may be received and/or accessed 412, for example retrieved from memory, either locally or on a PACS server, cloud, etc. A segmentation mask identifying a high uptake organ within the 3D functional image may be received and/or accessed as well 414. A segmentation mask, for example, may be produced from a medical image by automated image processing, manual selection and delineation by a user (e.g., a radiologist), or combinations thereof. For example, various approaches for automated segmentation of anatomical organs and tissue regions, described herein in Section B, may be used to determine a segmentation mask. Using organ boundaries of the high uptake organ from the segmentation mask, a local background intensity within the high uptake organ can be determined 416 from the 3D functional image. Using the determined local background intensity, 3D hotspot volumes within the 3D functional image corresponding to the high uptake organ may be determined 418. Hotspots may be further rendered and displayed 420, for example, as graphical shapes overlaid on medical images.

In certain embodiments, a local background intensity value associated with background uptake in a high-uptake organ comprises is determined as a mean and/or a standard deviation of the uptake value in the organ. In certain embodiments, a local background intensity value is determined by fitting a multi-component mixture model to intensities of voxels within a VOI corresponding to the high uptake organ. For example, a mean and a standard deviation of local background intensity in an organ is determined by fitting a two component Gaussian Mixture model to the organ intensity values. For example, a two component Gaussian mixture model may have the following form:

$$f ( x ) = {\sum }_{i = 1}^{2} {w}_{i} G ( x ; {μ}_{i} , {σ}_{i} )$$

where μᵢ is the mean, σᵢ is the standard deviation and wi is the weight of the i^{th} component and G is the Gaussian function. In certain embodiments, a mean (μ) and a standard deviation (σ) of a component with the largest weight, wᵢ, (e.g., a major mode) is chosen as an estimate of a mean and a standard deviation, respectively, of local background intensity corresponding to normal background uptake within the organ. A smaller component may be assumed to encapsulate abnormally low uptake (e.g., due to misalignment between a medical image and organ segmentation, and/or due to the cysts in the organ causing abnormally low uptake).

In certain embodiments, sub-regions within the high-uptake organ that represent locations of potential hotspots are detected by thresholding organ intensity values and selecting regions with intensities above a detection threshold detection value. In certain embodiments, a detection threshold detection value is determined based on (e.g., as a function of) the local background intensity value, for example based on a mean and standard deviation of a major mode determined via fitting a multi-component mixture model as described herein (e.g., as th=μ+4σ). In certain embodiments, an initial set of preliminary sub-regions are detected, and filtered via one or more selection criteria. One or more selection criteria may include (1) a degree of overlap (e.g., absolute volume, volumetric percentage) between a given one of the one or more preliminary sub-regions and a region (e.g., 3D volume of interest) of the 3D functional image corresponding to the high-uptake organ; (2) a degree of overlap (e.g., absolute volume, volumetric percentage) between a given one of the one or more preliminary sub-regions and a region (e.g., a 3D volume) of the 3D functional image corresponding to at least one other organ or tissue region [e.g., a neighboring organ, an organ with high physiological uptake, an organ known to “bleed over” (e.g., distort uptake values of a neighboring organ due to a misalignment between a functional image and a segmentation mask) into the high uptake organ, e.g., kidney]; (3 a minimum (e.g., hotspot) volume (e.g., a minimum number of voxels; e.g., a minimum corresponding physical volume) [e.g., such that only preliminary sub-regions having volumes greater than or equal to the minimum (e.g., hotspot) volume are selected] [e.g., wherein the minimum hotspot volume is at least 2 (e.g., 1, 5, 10) voxels] (e.g., to avoid noise induced hotspots).

In certain embodiments, hotspots are segmented by sub-dividing a VOI within the functional image corresponding to the high uptake organ into one or more sub-segments, for example using a watershed algorithm. Intensities within the VOI may be smoothed prior to sub-dividing it and peaks detected and used as basins in the watershed algorithm. Smoothing may avoid detecting noise as peaks. These determined sub-segments may then be compared with the detected sub-regions representing potential hotspots, to identify, for each preliminary sub-region, a subsegment that it overlaps with. A 3D hotspot volume corresponding to a particular sub-region (e.g., a j^{th} sub-region and corresponding hotspot volume) may be segmented using an individual segmentation threshold value (tsⱼ), determined based on (e.g., as a maximum of) the detection threshold value and a measure of intensity within the particular sub-region (e.g., a particular percentage, e.g., 60%, of the SUV max). For example, for a j^{th} sub-region and corresponding 3D hotspot volume to be segmented, a corresponding individual segmentation threshold may be determined via,

$${ts}_{j} = max ( μ + 4 σ \text{?} 0.6 \cdot SUV {max}_{j} ) \text{?}$$

$$\text{?} \text{indicates text missing or illegible when filed}$$

In certain embodiments, a 3D hotspot volume is segmented using a flood fill algorithm originating in the SUVmax of the detected hotspot, including values that are above the threshold tsⱼ, and are located in the subsegment(s) that the original sub-region (that represents the detected hotspot) overlaps with (e.g., the hotspot segmentation is not allowed to spread into subsegments that were not previously overlapped when the hotspot was detected).

TABLE 1
Certain Tissue Regions (*Prostate may, optionally, be segmented if
present - may be absent if patient has, e.g., undergone radical
prostatectomy, or may not segmented in any case,
in certain embodiments).
Organs/Bones

Right and Right Lung
Left and Right Femur
Left and Right Hip Bone
Urinary bladder
Sacrum and coccyx
Liver
Spleen
Left and Right Kidney
Left Side and Right Side Ribs 1-12
Left and Right Scapula
Left and Right Clavicle
Cervical vertebrae
Thoracic vertebrae 1-12
Lumbar vertebrae 1-5
Sternum
Aorta, thoracic part
Aorta, abdominal part
Prostate*

In certain embodiments, additionally or alternatively, hotspots may be classified as belonging to one or more lesion sub-types. In certain embodiments, lesion sub-type classifications may be made by comparing hotspot locations with classes of anatomical regions. For example, in certain embodiments a miTNM classification scheme may be used, where hotspots are labeled as belonging to one of three classes—miT, miN, or miM—based on whether they represent lesions located within a prostate (miT), pelvic lymph node (miN), or distant metastases (miM). In certain embodiments, a five class version of the miTNM scheme may be used, with distant metastases further divided into three sub classes—miMb for bone metastases, miMa for lymph metastases, and miMc for other soft tissue metastases.

For example, in certain embodiments, hotspots located within a prostate are labeled as belonging to class “T” or “miT”, e.g., representing local tumor. In certain embodiments, hotspots located outside a prostate, but within a pelvic region are labeled as class “N” or “miN”. In certain embodiments, for example as described in U.S. application Ser. No. 17/959,357, filed Oct. 4, 2022, entitled “Systems and Methods for Automated Identification and Classification of Lesions in Local Lymph and Distant Metastases,” published as U.S. 2023/0115732 A1 on Apr. 13, 2023, the content of which is incorporated herein by reference in its entirety, a pelvic atlas may be registered to identify boundaries of a pelvic region and/or various sub-regions therein, for purposes of identifying pelvic lymph node lesions. A pelvic atlas may, for example, include boundaries of a pelvic region and/or a planar reference (e.g., a plane passing through an aorta-bifurcation) which hotspot locations can be compared to (e.g., such that hotspots located outside the pelvic region and/or above the planar reference passing through an aorta bifurcation are labeled as “M” or “miM”—e.g., distant metastases). In certain embodiments, distant metastases may be classified as lymph (miMa), bone (miMb), or visceral (miMc) based on a comparison of hotspot locations with an anatomical segmentation map. For example, hotspots located within one or more bones (e.g., and outside a pelvic region) may be labeled as distant metastases, hotspots located within one or more segmented organs or a subset of organs (e.g., brain, lung, liver, spleen, kidneys) may be labeled as visceral (miMc) distant metastases, and remaining hotspots located outside a pelvic region labeled as distant lymph metastases (miMa).

Additionally or alternatively, in certain embodiments, hotspots may be assigned an miTNM class based on a determination that they are located within a particular anatomical region, for example based on a table such as Table 2, where each column corresponds to a particular miTNM label (first row indicating the particular miTNM class) and includes, in rows two and below, particular anatomical regions associated with each miTNM class. In certain embodiments, a hotspot can be assigned as being located within a particular tissue region listed in Table 2 based on a comparison of the hotspot's location with an anatomical segmentation map, allowing for an automated miTNM class assignment.

TABLE 2
An Example List of Tissue Regions Corresponding to Five
Classes in a Lesion Anatomical Labeling Approach.

| | | Pelvic lymph | | |
|---|---|---|---|---|
| Bone | Lymph nodes | nodes | Prostate | Visceral |
| Mb | Ma | N | T | Mc |
| Skull | Cervical | Template right | Prostate | Brain |
| Thorax | Supraclavicular | Template left | | Neck |
| Vertebrae | Axillary | Presacral | | Lung |
| lumbar | | | | |
| Vertebrae | Mediastinal | Other, pelvic | | Esophageal |
| thoracic | | | | |
| Pelvis | Hilar | | | Liver |
| Extremities | Mesenteric | | | Gallbladder |
| | Elbow | | | Spleen |
| | Popliteal | | | Pancreas |
| | Peri-/para-aortic | | | Adrenal |
| | Other, non- | | | Kidney |
| | pelvic | | | |
| | | | | Bladder |
| | | | | Skin |
| | | | | Muscle |
| | | | | Other |

In certain embodiments, hotspots may be further classified in terms of their anatomical location and/or lesion sub-type. For example, in certain embodiments, hotspots identified as located in pelvic lymph (miN) may be identified as belonging to a particular pelvic lymph node sub-region, such as one of a left/right internal iliac, a left or right external iliac, a left or right common iliac, a left or right obturator, a presacral region, or other pelvic region. In certain embodiments, distant lymph node metastases (miMa) may be classified as retroperitoneal (RP), supradiaphragmatic (SD), or other extrapelvic (OE). Approaches for regional (miN) and distant (miMa) lymph metastases classifications may include registration of pelvic atlas images and/or identification of various whole body landmarks, which are described in further detail in U.S. application Ser. No. 17/959,357, filed Oct. 4, 2022, entitled “Systems and Methods for Automated Identification and Classification of Lesions in Local Lymph and Distant Metastases,” published as U.S. 2023/0115732 A1 on Apr. 13, 2023, the content of which is incorporated herein by reference in its entirety.

### D. Uptake Region Quantification Metrics

In certain embodiments, detected—e.g., identified and segmented—uptake regions, such as hotspots, may be characterized via various individual quantification metrics. In particular, for a particular individual uptake region, such as a hotspot, uptake region quantification metrics can be used to quantify a measure of size (e.g., 3D volume) and/or intensity of the particular uptake region in a manner that is indicative of a size and/or level of radiopharmaceutical uptake within the (e.g., potential) underlying physical lesion that the particular uptake region (e.g., hotspot) represents. Accordingly, individual uptake region quantification metrics may convey, for example to a physician or radiologist, a likelihood that an uptake region (e.g., hotspot) appearing in an image represents a true underlying physical lesion and/or convey a likelihood or level of malignancy thereof (e.g., allowing to differentiate between benign and malignant lesions).

In certain embodiments, image segmentation, lesion detection, and characterization techniques as described herein are used to determine, for each of one or more medical images, a corresponding set of one or more uptake regions, such as a hotspot set. For example, as described herein, image segmentation techniques may be used to determine, for each hotspot detected in a particular image, a particular 3D volume—a 3D hotspot volume—representing and/or indicative of a volume (e.g., 3D location and extent) of a potential underlying physical lesion within the subject. Each uptake region, in turn, comprises a set of image voxels, each having a particular intensity value.

Once determined, a set of uptake regions may be used to compute one or more quantification metrics for each individual uptake region. Individual uptake region quantification metrics may be computed according to various methods and formulae described herein, for example below. In the description below, the variable L is used to refer to a set of uptake regions detected with a particular image, with L={1, 2, . . . , l, . . . , N_{L}} representing a set of N_{L} (i.e., N_{L} being the number of uptake regions, such as hotspots) uptake regions detected within an image and the variable l indexing the l^{th} uptake region. As described herein, each uptake region corresponds to a particular 3D volume within an image, with Rₗ denoting the volume of the l^{th} uptake region.

Uptake region quantification metrics may be presented to a user via a GUI and/or a (e.g., automatically or semi-automatically) generated report. As described in further detail herein, individual uptake region quantification metrics may include uptake region intensity metrics and uptake region volume metrics (e.g., lesion volume) that quantify an intensity and size, respectively of a particular uptake region and/or underlying lesion it represents. Uptake region intensity and size may, in turn, be indicative of a level of radiopharmaceutical uptake within, and size of, respectively, an underlying physical lesion within the subject.

### D.i. Uptake Region Intensity Metrics

In certain embodiments, an uptake quantification metric is or comprises an uptake region intensity metric or uptake intensity metric that quantifies an intensity of an individual uptake region, such as a 3D hotspot volume. Uptake intensity metrics may be computed based on individual voxel intensities within identified uptake regions. For example, for a particular uptake region, a value of an uptake intensity metric may be computed as a function of at least a portion (e.g., a particular subset, e.g., all) of that hotspot's voxel intensities. Uptake intensity metrics may include, without limitation, metrics such as a maximum uptake intensity, a mean uptake intensity, and peak uptake intensity, and the like. As with voxel intensities in nuclear medicine images, in certain embodiments uptake intensity metrics may represent (e.g., be in units of) SUV values.

In certain embodiments, a value of a particular uptake intensity metric is computed, for a subject uptake region, based on (e.g., as a function of) that subject uptake region's voxel intensities alone, e.g., and not based on intensities of other image voxels outside the subject uptake region.

For example, an uptake intensity metric may be a maximum uptake intensity (e.g., SUV), or “SUV-max,” computed as a maximum voxel intensity (e.g., SUV or uptake) within an uptake region (e.g., within a 3D hotspot volume). In certain embodiments, a maximum uptake intensity may be computed according to equations (la), (1b), or (1c), below.

$${Q}_{max} ( l ) = \text{?} ( {q}_{i} ) \qquad ( 1 a )$$

$${SUV}_{max} ( l ) = \text{?} ( {SUV}_{i} ) \qquad ( 1 b )$$

$$SUV = max ( UptakeInVoxel ∈ lesion volume ) \qquad ( 1 c )$$

$$\text{?} \text{indicates text missing or illegible when filed}$$

where, in equations (1a) and (1b) l represents a particular (e.g., l^{th}) uptake region, as described above, qᵢ is the intensity of voxel i and i∈Rₗ is the set of voxels within the particular uptake region, Rₗ. In equation (1b), SUVᵢ indicates a particular unit—standardized uptake value (SUV)—of voxel intensity, as described herein.

In certain embodiments, an uptake intensity metric may be a mean uptake region intensity (e.g., SUV), or “SUV-mean,” and may be computed as a mean over all voxel intensities (e.g., SUV or uptake) within an uptake region. In certain embodiments, a mean uptake region intensity may be computed according to equations (2a), (2b), or (2c) below.

$${Q}_{mean} ( l ) = \text{?} ( {q}_{i} ) = \frac{1}{\text{?}} \text{?} {q}_{i} \qquad ( 2 a )$$

$${SUV}_{mean} ( l ) = \text{?} ( {SUV}_{i} ) = \frac{1}{\text{?}} \text{?} {SUV}_{i} \qquad ( 2 b )$$

$${SUV}_{mean} = \text{?} \frac{UptakeInVoxel}{\text{?}} \qquad ( 2 c )$$

$$\text{?} \text{indicates text missing or illegible when filed}$$

where nₗ is the number of individual voxels within a particular uptake region.

In certain embodiments, an uptake region intensity metric may be a peak uptake region intensity (e.g., SUV), or “SUV-peak,” and may be computed as a mean over intensities of the voxels (e.g., SUV or uptake) whose midpoints are located within a (e.g., pre-defined) particular distance (e.g., within 5 mm) of the midpoint of the uptake region voxel where the maximum intensity (e.g., SUV-max) is located within an uptake region, and, accordingly, may be computed according to equations (3a)-(3c) below.

$${Q}_{peak} ( l ) = \frac{1}{\text{?}} {{\sum }_{i :}}_{dist ( {i}_{max} , i ) \leq d} {q}_{i} \qquad ( 3 a )$$

$${SUV}_{peak} ( l ) = \frac{1}{\text{?}} {{\sum }_{i :}}_{dist ( {i}_{max} , i ) \leq d} {SUV}_{i} \qquad ( 3 b )$$

$${SUV}_{peak} = \frac{1}{\text{?}} \text{?} {UptakeInVoxel}_{i} \qquad ( 3 c )$$

$$\text{?} \text{indicates text missing or illegible when filed}$$

where i: dist(iₘₐₓ, i)≤d is the set of (uptake region) voxels having a mid-point within a distance, d, from voxel iₘₐₓ, which is the maximum intensity voxel within the uptake region (e.g., Qₘₐₓ(l)=qᵢ₋ₘₐₓ.

### D.ii. Lesion Index Metrics

In certain embodiments, an uptake region intensity metric is individual lesion index value that maps an intensity of voxels within a particular 3D hotspot volume to a value on a standardized scale. Such lesion index values are described in further detail in PCT/EP2020/050132, filed Jan. 6, 2020, and PCT/EP2021/068337, filed Jul. 2, 2021, the content of each of which is hereby incorporated by reference in its entirety. Calculation of lesion index values may include calculation of reference intensity values within particular reference tissue regions, such as an aorta portion (also referred to as blood pool) and/or a liver.

For example, in one particular implementation, a first, blood-pool, reference intensity value is determined based on a measure of intensity (e.g., a mean SUV) within an aorta region and a second, liver, reference intensity value is determined based on a measure of intensity (e.g., a mean SUV) within a liver region. As described in further detail, for example in PCT/EP2021/068337, filed Jul. 2, 2021, the content of which is incorporated herein by reference in its entirety, calculation of reference intensities may include approaches such as identifying reference volumes (e.g., an aorta or portion thereof; e.g., a liver volume) within a functional image, such as a PET or SPECT image, eroding and/or dilating certain reference volumes, e.g., to avoid include voxels on the edge of a reference volume, and selecting subsets of reference voxel intensities, based on modeling approaches, e.g., to account for anomalous tissue features, such as cysts and lesions, within a liver. In certain embodiments, a third reference intensity value may be determined, either as a multiple (e.g., twice) of a liver reference intensity value, or based on an intensity of another reference tissue region, such as a parotid gland.

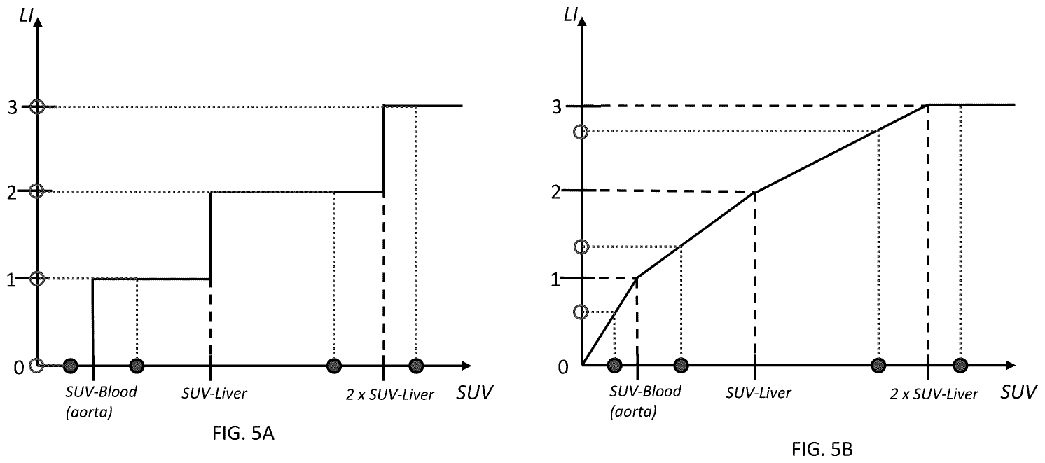

In certain embodiments, uptake region intensities may be compared with one or more reference intensity values to determine a lesion index as a value on a standardized scale, which facilitates comparison across different images. For example, FIGS. 5A and 5B illustrate approaches for assigning uptake regions a lesion index value ranging from 0 to 3. In the approach shown in FIGS. 5A and 5B, a blood-pool (aorta) intensity value is assigned a lesion index of 1, a liver intensity value is assigned a lesion 2, and a value of twice the liver intensity is assigned a lesion index of 3. A lesion index for a particular uptake region can be determined by first computing a value of an initial uptake region intensity metric for the particular uptake region, such as a mean uptake region intensity (e.g., Qₘₑₐₙ(l) or SUVₘₑₐₙ) and comparing the value of the initial uptake region intensity metric with the reference intensity values. For example, the value of the initial hotspot intensity metric may fall within one of four ranges—[0, SUV_{blood}], (SUV_{blood}, SUVₗᵢᵥₑᵣ], (SUVₗᵢᵥₑᵣ, 2×SUVₗᵢᵥₑᵣ], and greater than 2×SUVₗᵢᵥₑᵣ (e.g., (2×SUVₗᵢᵥₑᵣ, ∞)). In certain embodiments, an uptake region may be assigned a lesion index value based on a step function, e.g., as shown in FIG. 5A. In certain embodiments, as shown in FIG. 5B a lesion index value can then be computed for the particular uptake region based on (i) the value of the initial uptake region intensity metric and (ii) a linear interpolation according to the particular range in which the value of the initial uptake region intensity metric falls, as illustrated in FIG. 5B, where the filled and open dots on the horizontal (SUV) and vertical (LI) axes illustrate example values of initial hotspot intensity metrics and resultant lesion index values, respectively. In certain embodiments, if SUV references for either liver or aorta cannot be calculated, or if the aorta value is higher than the liver value, the lesion index will not be calculated and will be displayed as ‘-’.

A lesion index value according to the mapping scheme described above and illustrated in FIG. 5B may, for example, be computed as shown in equation (4), below.

$$\text{?} ( l ) = \{ \begin{matrix}{f}_{1} ( {SUV}_{mean} ( l ) ) , & {SUV}_{mean} ( l ) \leq {SUV}_{aorta} \\ {f}_{2} ( {SUV}_{mean} ( l ) ) , & {SUV}_{aorta} \leq {SUV}_{mean} ( l ) \leq {SUV}_{liver} \\ {f}_{3} ( {SUV}_{mean} ( l ) ) , & {SUV}_{liver} \leq {SUV}_{mean} ( l ) \leq 2 \times {SUV}_{liver} \\ 3 , & 2 \times {SUV}_{liver} \leq {SUV}_{mean} ( l )\end{matrix} \qquad ( 4 )$$

$$\text{?} \text{indicates text missing or illegible when filed}$$

where f₁ f₂ and f₃ are linear interpolations between the respective spans in equation (4).

### D.iii. Uptake Region/Lesion Volume

In certain embodiments, an uptake region quantification metric may be a volume metric, such as a lesion volume, Qᵥₒₗ, which provides a measure of size (e.g., volume) of an underlying physical lesion that an uptake region represents. A lesion volume may, in certain embodiments, computed as shown in equations (5a) and (5b), below.

$${Q}_{vol} ( l ) = \text{?} {v}_{i} \qquad ( 5 a )$$

$${Q}_{vol} ( l ) = v \times \text{?} \qquad ( 5 b )$$

$$\text{?} \text{indicates text missing or illegible when filed}$$

where in equation (5a), vᵢ is a volume of an i^{th} voxel, and equation (5b) assumes a uniform voxel volume, v, and as before nₗ is a number of voxels in a particular hotspot volume, l. In certain embodiments, a voxel volume is computed as v=δx×δy×δz, where δx, δy, and δz are grid spacing (e.g., in millimeters, mm) in x, y, and z. In certain embodiments, a lesion volume has units of milliliters (ml).

### E. Automated Evaluation of Cancer Staging Scores

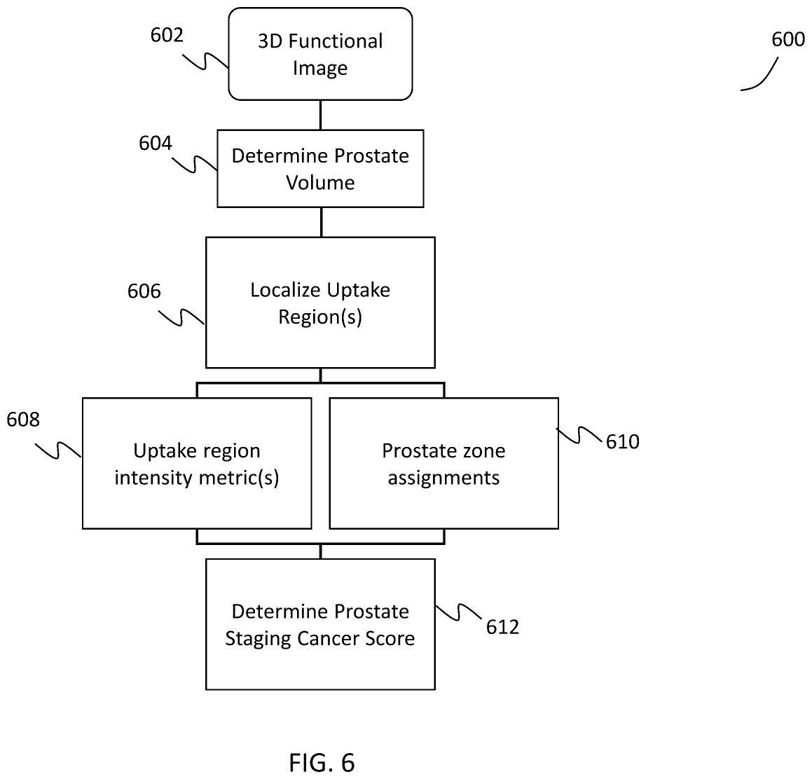

Turning to FIG. 6, in certain embodiments, various approaches for localizing 3D volumes of interest (VOIs) within images, e.g., corresponding to particular organs, and identifying and characterizing uptake regions (e.g., hotspots) corresponding to potential lesions, e.g., including, but not limited to, those described herein, may be used to determine cancer staging scores in an automated fashion. For example, in certain embodiments, in an exemplary process 600 for automatically determining a prostate cancer staging score may utilize, and receive, a 3D functional image 602, such as a PET or SPECT image of a subject. A prostate volume, that corresponds to a prostate within the subject, may be determined (e.g., identified) 604 within the 3D functional image 602. A prostate volume within a 3D functional image may, for example, be determined using various anatomical segmentation techniques described herein, for example by segmenting a 3D anatomical image, such as a CT image, that is co-aligned with the 3D functional image to determine a segmentation mask representing a region of the CT image determined to correspond to the prostate. The segmentation mask can then be mapped to the 3D functional image to identify, as the prostate volume, a corresponding region within the 3D functional image.

In certain embodiments, uptake regions that represent underlying physical lesions within the subject are localized 606 within the 3D functional image. As described herein, uptake regions may be hotspots, and may be localized—e.g., detected and/or segmented—using various techniques, such as machine learning based techniques, as described herein. In certain embodiments, localized uptake regions are limited to those determined represent underlying physical lesions and/or radiopharmaceutical uptake within the subject's prostate and/or its vicinity. These prostate uptake regions may be identified as those having volumes that overlap, e.g., at least in part, with, and/or are entirely within, the prostate volume.

In certain embodiments, for values of uptake region intensity metric(s) are determined for each localized (e.g., prostate) uptake region 608. For example, for each uptake region intensity metric, a value of a maximum, mean, median, peak, etc., intensity metric may be determined, e.g., as described herein. In certain embodiments, uptake region intensity metrics determined for each uptake region include values of lesion indices, determined, for example, using reference intensity values, such as those of a liver and/or aorta region, for example as described herein. In certain embodiments, for example where a radiopharmaceutical used to obtain the 3D functional image is or comprises a PSMA binding agent, a lesion index may be referred to as a PSMA expression score.

In certain embodiments, a spatial location of each uptake region is analyzed and compared with one or more prostate zones 610, and a set of assigned prostate zones determined, to reflect a spatial location and/or distribution of intensities within the uptake region. For example, a prostate may be divided into 4 zones according to their biological function. For example, a prostate and its immediate vicinity may be subdivided into a central zone (CZ) (e.g., a zone that surrounds the ejaculatory ducts and comprises about 25% of a prostate's total mass), a transitional zone (TZ) (e.g., a part of the prostate that surrounds the urethra), and a peripheral zone (PZ) (e.g., a zone situated toward a back of the gland, where a majority of the glandular tissue sits. In certain embodiments, other zones, such as a fibromuscular zone (FZ) (e.g., anterior zone), may be used as well. In certain embodiments, a FZ may be challenging to identify on a CT image, and not included in a model. In certain embodiments, a ureter zone (UZ) may also be included. A reference model, e.g., an atlas image, with boundaries of the particular prostate zones may be aligned to the 3D functional image, and a spatial extent of each uptake region compared with the so aligned reference model, to determine which of the various prostate zones the uptake region is located within and/or overlaps with. An uptake region may be associated with a set of assigned zones that it overlaps with, for example a single zone or multiple zones.

In certain embodiments, once determined, the values of the uptake region intensity metric(s) and the prostate zone assignments may be used to determine a prostate cancer staging score 612.

### F. Computer System and Network Architecture

Certain embodiments described herein make use of computer algorithms in the form of software instructions executed by a computer processor. In certain embodiments, the software instructions include a machine learning module, also referred to herein as artificial intelligence software. As used herein, a machine learning module refers to a computer implemented process (e.g., a software function) that implements one or more specific machine learning techniques, e.g., artificial neural networks (ANNs), e.g., convolutional neural networks (CNNs), e.g., recursive neural networks, e.g., recurrent neural networks such as long short-term memory (LSTM) or Bilateral long short-term memory (Bi-LSTM), random forest, decision trees, support vector machines, and the like, in order to determine, for a given input, one or more output values.

In certain embodiments, machine learning modules implementing machine learning techniques are trained, for example using datasets that include categories of data described herein. Such training may be used to determine various parameters of machine learning algorithms implemented by a machine learning module, such as weights associated with layers in neural networks. In certain embodiments, once a machine learning module is trained, e.g., to accomplish a specific task, values of determined parameters are fixed and the (e.g., unchanging, static) machine learning module is used to process new data (e.g., different from the training data) and accomplish its trained task without further updates to its parameters (e.g., the machine learning module does not receive feedback and/or updates). In certain embodiments, machine learning modules may receive feedback, and such feedback may be used as additional training data to dynamically update the machine learning module. In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions of an ANN module may be carried out via specialized hardware (e.g., via an application specific integrated circuit (ASIC)).

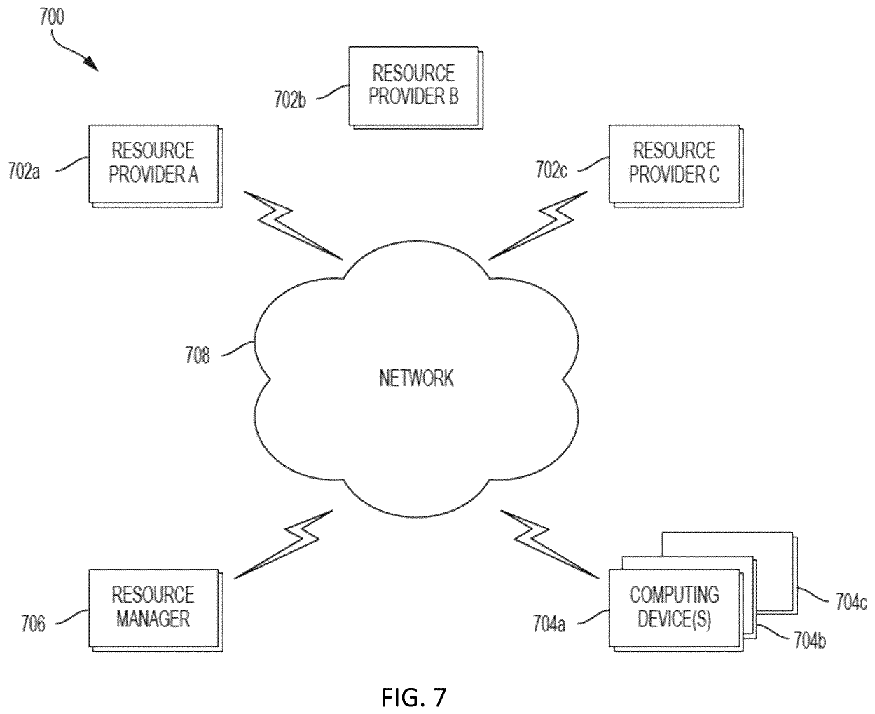

As shown in FIG. 7, an implementation of a network environment 700 for use in providing systems, methods, and architectures as described herein is shown and described. In brief overview, referring now to FIG. 7, a block diagram of an exemplary cloud computing environment 700 is shown and described. The cloud computing environment 700 may include one or more resource providers 702a, 702b, 702c (collectively, 702). Each resource provider 702 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 702 may be connected to any other resource provider 702 in the cloud computing environment 700. In some implementations, the resource providers 702 may be connected over a computer network 708. Each resource provider 702 may be connected to one or more computing device 704a, 704b, 704c (collectively, 704), over the computer network 708.

The cloud computing environment 700 may include a resource manager 706. The resource manager 706 may be connected to the resource providers 702 and the computing devices 704 over the computer network 708. In some implementations, the resource manager 706 may facilitate the provision of computing resources by one or more resource providers 702 to one or more computing devices 704. The resource manager 706 may receive a request for a computing resource from a particular computing device 704. The resource manager 706 may identify one or more resource providers 702 capable of providing the computing resource requested by the computing device 704. The resource manager 706 may select a resource provider 702 to provide the computing resource. The resource manager 706 may facilitate a connection between the resource provider 702 and a particular computing device 704. In some implementations, the resource manager 706 may establish a connection between a particular resource provider 702 and a particular computing device 704. In some implementations, the resource manager 706 may redirect a particular computing device 704 to a particular resource provider 702 with the requested computing resource.

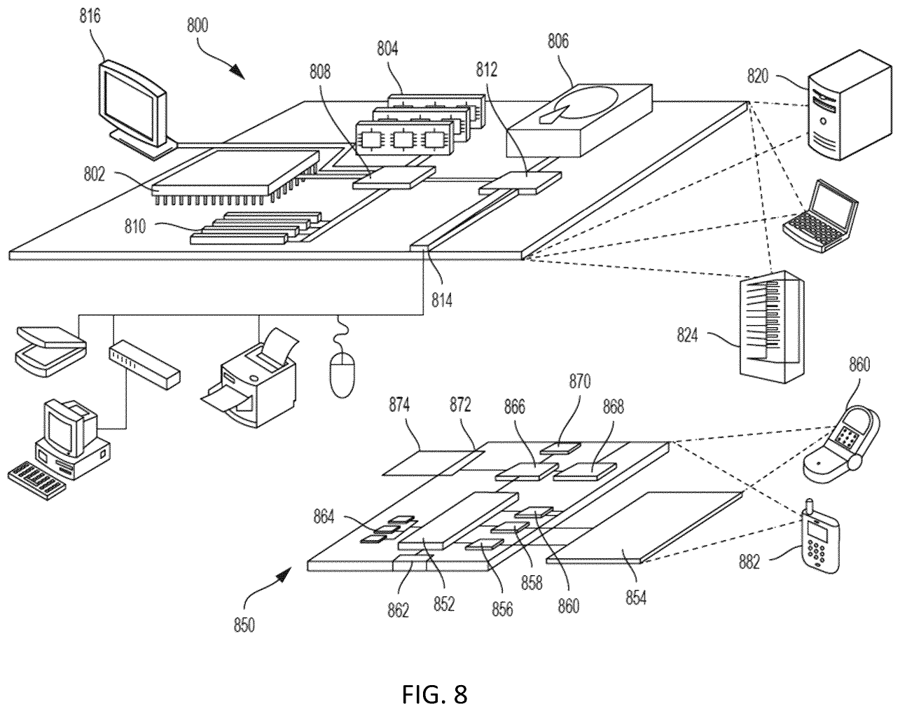

FIG. 8 shows an example of a computing device 800 and a mobile computing device 850 that can be used to implement the techniques described in this disclosure. The computing device 800 is intended to represent various forms of digital computers, such as laptops, desktops, workstations, personal digital assistants, servers, blade servers, mainframes, and other appropriate computers. The mobile computing device 850 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 800 includes a processor 802, a memory 804, a storage device 806, a high-speed interface 808 connecting to the memory 804 and multiple high-speed expansion ports 810, and a low-speed interface 812 connecting to a low-speed expansion port 814 and the storage device 806. Each of the processor 802, the memory 804, the storage device 806, the high-speed interface 808, the high-speed expansion ports 810, and the low-speed interface 812, are interconnected using various busses, and may be mounted on a common motherboard or in other manners as appropriate. The processor 802 can process instructions for execution within the computing device 800, including instructions stored in the memory 804 or on the storage device 806 to display graphical information for a GUI on an external input/output device, such as a display 816 coupled to the high-speed interface 808. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 804 stores information within the computing device 800. In some implementations, the memory 804 is a volatile memory unit or units. In some implementations, the memory 804 is a non-volatile memory unit or units. The memory 804 may also be another form of computer-readable medium, such as a magnetic or optical disk.

The storage device 806 is capable of providing mass storage for the computing device 800. In some implementations, the storage device 806 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 802), perform one or more methods, such as those described above. The instructions can also be stored by one or more storage devices such as computer-or machine-readable mediums (for example, the memory 804, the storage device 806, or memory on the processor 802).

The high-speed interface 808 manages bandwidth-intensive operations for the computing device 800, while the low-speed interface 812 manages lower bandwidth-intensive operations. Such allocation of functions is an example only. In some implementations, the high-speed interface 808 is coupled to the memory 804, the display 816 (e.g., through a graphics processor or accelerator), and to the high-speed expansion ports 810, which may accept various expansion cards (not shown). In the implementation, the low-speed interface 812 is coupled to the storage device 806 and the low-speed expansion port 814. The low-speed expansion port 814, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 800 may be implemented in a number of different forms, as shown in the figure. For example, it may be implemented as a standard server 820, or multiple times in a group of such servers. In addition, it may be implemented in a personal computer such as a laptop computer 822. It may also be implemented as part of a rack server system 824. Alternatively, components from the computing device 800 may be combined with other components in a mobile device (not shown), such as a mobile computing device 850. Each of such devices may contain one or more of the computing device 800 and the mobile computing device 850, and an entire system may be made up of multiple computing devices communicating with each other.

The mobile computing device 850 includes a processor 852, a memory 864, an input/output device such as a display 854, a communication interface 866, and a transceiver 868, among other components. The mobile computing device 850 may also be provided with a storage device, such as a micro-drive or other device, to provide additional storage. Each of the processor 852, the memory 864, the display 854, the communication interface 866, and the transceiver 868, are interconnected using various buses, and several of the components may be mounted on a common motherboard or in other manners as appropriate.

The processor 852 can execute instructions within the mobile computing device 850, including instructions stored in the memory 864. The processor 852 may be implemented as a chipset of chips that include separate and multiple analog and digital processors. The processor 852 may provide, for example, for coordination of the other components of the mobile computing device 850, such as control of user interfaces, applications run by the mobile computing device 850, and wireless communication by the mobile computing device 850.

The processor 852 may communicate with a user through a control interface 758 and a display interface 856 coupled to the display 854. The display 854 may be, for example, a TFT (Thin-Film-Transistor Liquid Crystal Display) display or an OLED (Organic Light Emitting Diode) display, or other appropriate display technology. The display interface 856 may comprise appropriate circuitry for driving the display 854 to present graphical and other information to a user. The control interface 858 may receive commands from a user and convert them for submission to the processor 852. In addition, an external interface 862 may provide communication with the processor, so as to enable near area communication of the mobile computing device 850 with other devices. The external interface 862 may provide, for example, for wired communication in some implementations, or for wireless communication in other implementations, and multiple interfaces may also be used.

The memory 864 stores information within the mobile computing device 850. The memory 864 can be implemented as one or more of a computer-readable medium or media, a volatile memory unit or units, or a non-volatile memory unit or units. An expansion memory 874 may also be provided and connected to the mobile computing device 850 through an expansion interface 872, which may include, for example, a SIMM (Single In Line Memory Module) card interface. The expansion memory 874 may provide extra storage space for the mobile computing device 850, or may also store applications or other information for the mobile computing device 850. Specifically, the expansion memory 874 may include instructions to carry out or supplement the processes described above, and may include secure information also. Thus, for example, the expansion memory 874 may be provide as a security module for the mobile computing device 850, and may be programmed with instructions that permit secure use of the mobile computing device 850. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 852), perform one or more methods, such as those described above. The instructions can also be stored by one or more storage devices, such as one or more computer-or machine-readable mediums (for example, the memory 864, the expansion memory 874, or memory on the processor 852). In some implementations, the instructions can be received in a propagated signal, for example, over the transceiver 868 or the external interface 862.

The mobile computing device 850 may communicate wirelessly through the communication interface 866, which may include digital signal processing circuitry where necessary. The communication interface 866 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 868 using a radio-frequency. In addition, short-range communication may occur, such as using a Bluetooth®, Wi-Fi™, or other such transceiver (not shown). In addition, a GPS (Global Positioning System) receiver module 870 may provide additional navigation-and location-related wireless data to the mobile computing device 850, which may be used as appropriate by applications running on the mobile computing device 850.

The mobile computing device 850 may also communicate audibly using an audio codec 860, which may receive spoken information from a user and convert it to usable digital information. The audio codec 860 may likewise generate audible sound for a user, such as through a speaker, e.g., in a handset of the mobile computing device 850. Such sound may include sound from voice telephone calls, may include recorded sound (e.g., voice messages, music files, etc.) and may also include sound generated by applications operating on the mobile computing device 850.

The mobile computing device 850 may be implemented in a number of different forms, as shown in the figure. For example, it may be implemented as a cellular telephone 880. It may also be implemented as part of a smart-phone 882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

### G. Imaging Agents

As described herein, a variety of radionuclide labelled PSMA binding agents may be used as radiopharmaceutical imaging agents for nuclear medicine imaging to detect and evaluate prostate cancer. In certain embodiments, certain radionuclide labelled PSMA binding agents are appropriate for PET imaging, while others are suited for SPECT imaging.

### G.i. PET Imaging Radionuclide Labelled PSMA Binding Agents

In certain embodiments, a radionuclide labelled PSMA binding agent is a radionuclide labelled PSMA binding agent appropriate for PET imaging.

In certain embodiments, a radionuclide labelled PSMA binding agent comprises [18F]DCFPyL (also referred to as PyL™; also referred to as DCFPyL-18F):

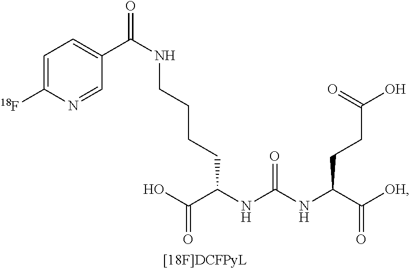

or a pharmaceutically acceptable salt thereof.

In certain embodiments, a radionuclide labelled PSMA binding agent comprises [18F]DCFBC:

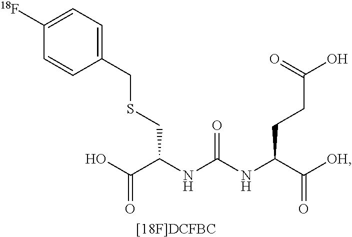

or a pharmaceutically acceptable salt thereof.

In certain embodiments, a radionuclide labelled PSMA binding agent comprises ⁶⁸Ga-PSMA-HBED-CC (also referred to as ⁶⁸Ga-PSMA-11):

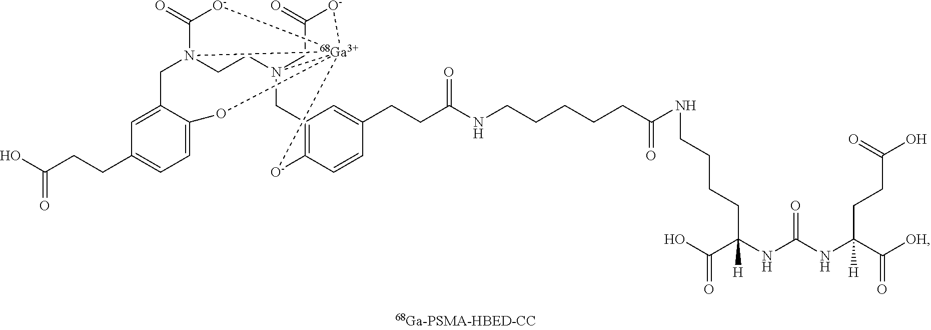

or a pharmaceutically acceptable salt thereof.

In certain embodiments, a radionuclide labelled PSMA binding agent comprises PSMA-617:

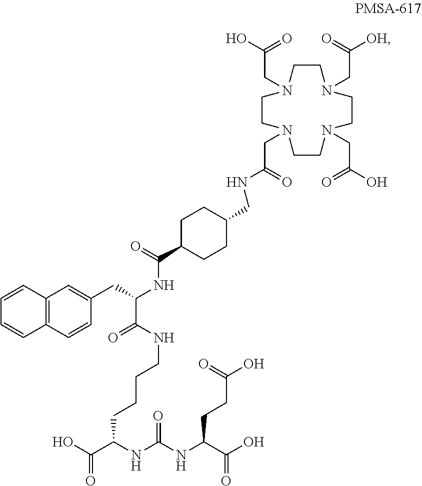 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

or a pharmaceutically acceptable salt thereof. In certain embodiments, the radionuclide labelled PSMA binding agent comprises ⁶⁸Ga-PSMA-617, which is PSMA-617 labelled with ⁶⁸Ga, or a pharmaceutically acceptable salt thereof. In certain embodiments, a radionuclide labelled PSMA binding agent comprises ¹⁷⁷Lu-PSMA-617, which is PSMA-617 labelled with ¹⁷⁷Lu, or a pharmaceutically acceptable salt thereof.

In certain embodiments, a radionuclide labelled PSMA binding agent comprises PSMA-I&T:

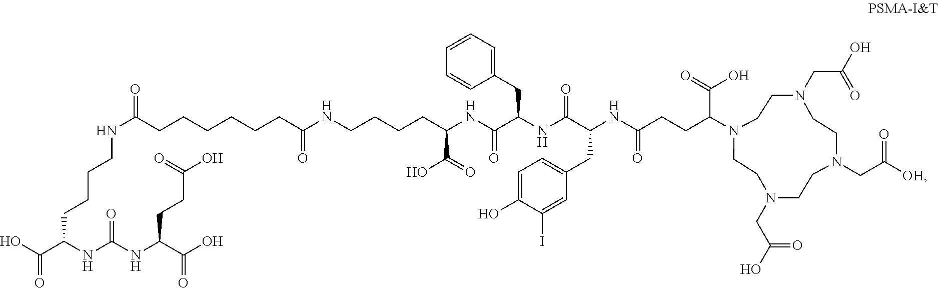 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

or a pharmaceutically acceptable salt thereof. In certain embodiments, a radionuclide labelled PSMA binding agent comprises ⁶⁸Ga-PSMA-I&T, which is PSMA-I&T labelled with ⁶⁸Ga, or a pharmaceutically acceptable salt thereof.

In certain embodiments, a radionuclide labelled PSMA binding agent comprises PSMA-1007:

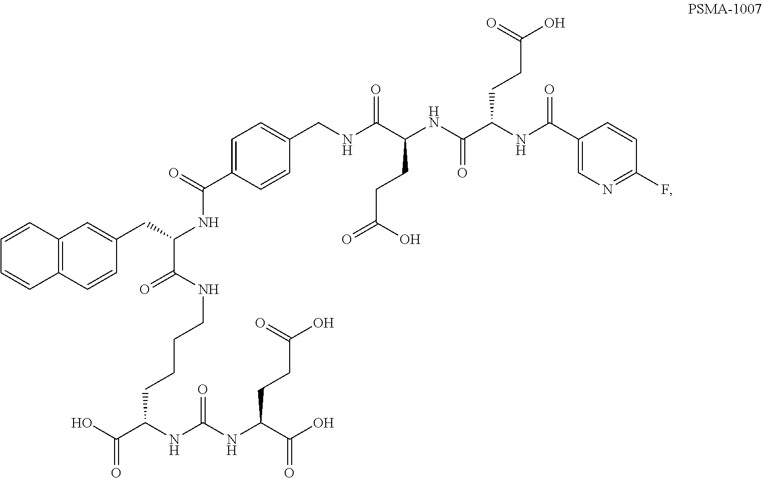 [SMILES: O=C(O)CC[C@H](NC(=O)N[C@@H](CCCCNC(=O)[C@H](Cc1ccc2ccccc2c1)NC(=O)c1ccc(CNC(=O)[C@H](CCC(=O)O)NC(=O)[C@H](CCC(=O)O)NC(=O)c2ccc(F)nc2)cc1)C(=O)O)C(=O)O]

or a pharmaceutically acceptable salt thereof. In certain embodiments, a radionuclide labelled PSMA binding agent comprises 18F-PSMA-1007, which is PSMA-1007 labelled with 18F, or a pharmaceutically acceptable salt thereof.

In certain embodiments, a radionuclide labeled PSMA binding agent comprises 18F-JK-PSMA-7:

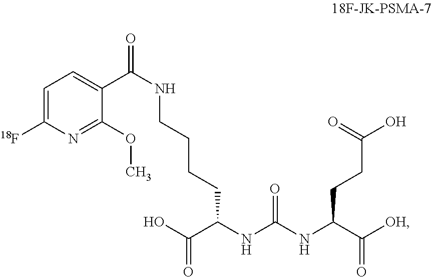 [SMILES: COc1nc([18F])ccc1C(=O)NCCCC[C@H](NC(=O)N[C@@H](CCC(=O)O)C(=O)O)C(=O)O]

or a pharmaceutically acceptable salt thereof.

In certain embodiments, a radionuclide labeled PSMA binding agent comprises (18F) rhPSMA-7.3 (e.g., POSLUMA®, also described at https://www.posluma.com/prescribing-information.pdf):

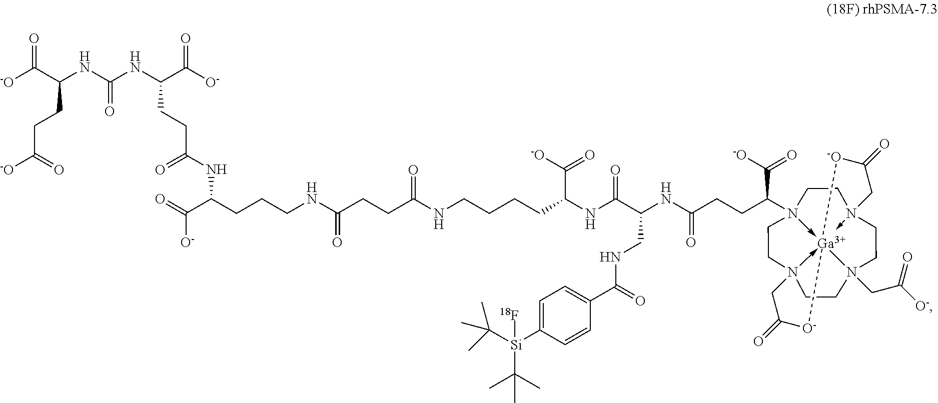

or a pharmaceutically acceptable salt thereof.

### G.ii. SPECT Imaging Radionuclide Labelled PSMA Binding Agents

In certain embodiments, a radionuclide labelled PSMA binding agent is a radionuclide labelled PSMA binding agent appropriate for SPECT imaging.

In certain embodiments, a radionuclide labelled PSMA binding agent comprises 1404 (also referred to as MIP-1404):

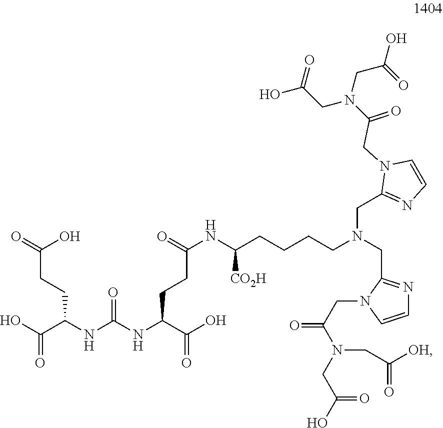 [SMILES: O=C(O)CC[C@H](NC(=O)N[C@@H](CCC(=O)N[C@@H](CCCCN(Cc1nccn1CC(=O)N(CC(=O)O)CC(=O)O)Cc1nccn1CC(=O)N(CC(=O)O)CC(=O)O)C(=O)O)C(=O)O)C(=O)O]

or a pharmaceutically acceptable salt thereof.

In certain embodiments, a radionuclide labelled PSMA binding agent comprises 1405 (also referred to as MIP-1405):

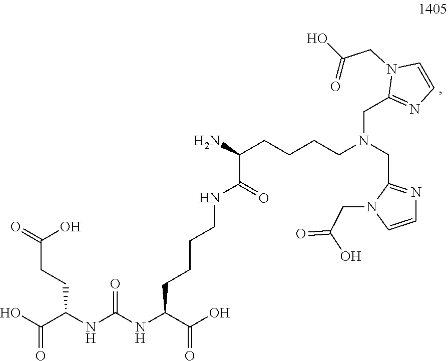 [SMILES: N[C@@H](CCCCN(Cc1nccn1CC(=O)O)Cc1nccn1CC(=O)O)C(=O)NCCCC[C@H](NC(=O)N[C@@H](CCC(=O)O)C(=O)O)C(=O)O]

or a pharmaceutically acceptable salt thereof.

In certain embodiments, a radionuclide labelled PSMA binding agent comprises 1427 (also referred to as MIP-1427):

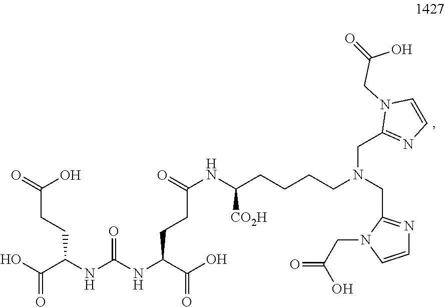 [SMILES: O=C(O)CC[C@H](NC(=O)N[C@@H](CCC(=O)N[C@@H](CCCCN(Cc1nccn1CC(=O)O)Cc1nccn1CC(=O)O)C(=O)O)C(=O)O)C(=O)O]

or a pharmaceutically acceptable salt thereof.

In certain embodiments, a radionuclide labelled PSMA binding agent comprises 1428 (also referred to as MIP-1428):

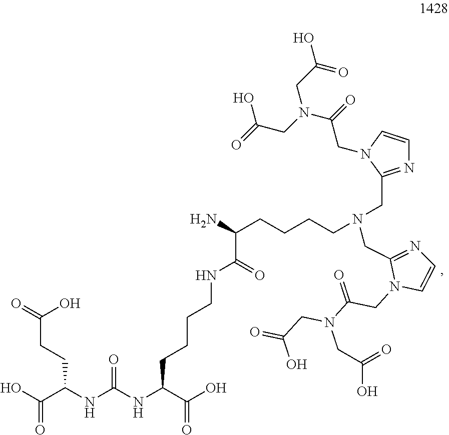 [SMILES: N[C@@H](CCCCN(Cc1nccn1CC(=O)N(CC(=O)O)CC(=O)O)Cc1nccn1CC(=O)N(CC(=O)O)CC(=O)O)C(=O)NCCCC[C@H](NC(=O)N[C@@H](CCC(=O)O)C(=O)O)C(=O)O]

or a pharmaceutically acceptable salt thereof.

In certain embodiments, a PSMA binding agent is labelled with a radionuclide by chelating it to a radioisotope of a metal [e.g., a radioisotope of technetium (Tc) (e.g., technetium-99m (99mTc)); e.g., a radioisotope of rhenium (Re) (e.g., rhenium-188 (188Re); e.g., rhenium-186 (186Re)); e.g., a radioisotope of yttrium (Y) (e.g., 90Y); e.g., a radioisotope of lutetium (Lu) (e.g., 177Lu); e.g., a radioisotope of gallium (Ga) (e.g., 68Ga; e.g., 67Ga); e.g., a radioisotope of indium (e.g., 111In); e.g., a radioisotope of copper (Cu) (e.g., 67Cu)].

In certain embodiments, 1404 is labelled with a radionuclide (e.g., chelated to a radioisotope of a metal). In certain embodiments, a radionuclide labelled PSMA binding agent comprises ^{99m}Tc-MIP-1404, which is 1404 labelled with (e.g., chelated to) ^{99m}Tc:

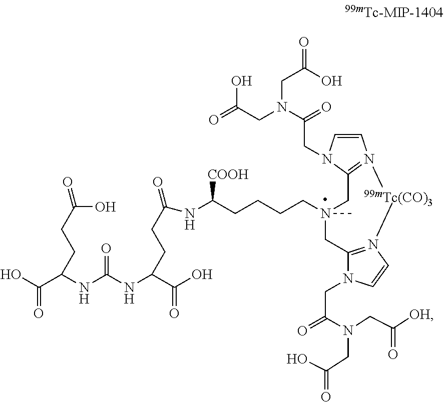

or a pharmaceutically acceptable salt thereof. In certain embodiments, 1404 may be chelated to other metal radioisotopes [e.g., a radioisotope of rhenium (Re) (e.g., rhenium-188 (¹⁸⁸Re); e.g., rhenium-186 (186Re)); e.g., a radioisotope of yttrium (Y) (e.g., 90Y); e.g., a radioisotope of lutetium (Lu) (e.g., 177Lu); e.g., a radioisotope of gallium (Ga) (e.g., 68Ga; e.g., 67Ga); e.g., a radioisotope of indium (e.g., 111In); e.g., a radioisotope of copper (Cu) (e.g., 67Cu)] to form a compound having a structure similar to the structure shown above for 99mTc-MIP-1404, with the other metal radioisotope substituted for 99mTc.

In certain embodiments, 1405 is labelled with a radionuclide (e.g., chelated to a radioisotope of a metal). In certain embodiments, a radionuclide labelled PSMA binding agent comprises ^{99m}Tc-MIP-1405, which is 1405 labelled with (e.g., chelated to) ^{99m}Tc:

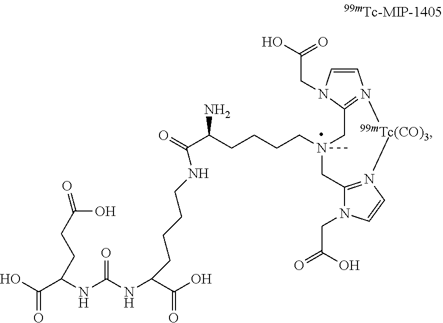

or a pharmaceutically acceptable salt thereof. In certain embodiments, 1405 may be chelated to other metal radioisotopes [e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu) (e.g., 177Lu); e.g., a radioisotope of gallium (Ga) (e.g., 68Ga; e.g., 67Ga); e.g., a radioisotope of indium (e.g., 111In); e.g., a radioisotope of copper (Cu) (e.g., 67Cu)] to form a compound having a structure similar to the structure shown above for 99mTc-MIP-1405, with the other metal radioisotope substituted for 99mTc.

In certain embodiments, 1427 is labelled with (e.g., chelated to) a radioisotope of a metal, to form a compound according to the formula below:

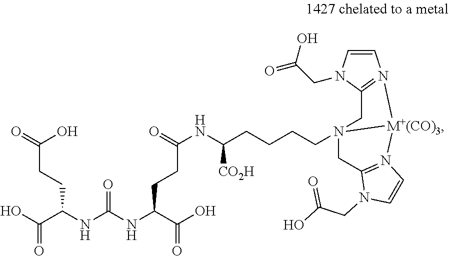

or a pharmaceutically acceptable salt thereof, wherein M is a metal radioisotope [e.g., a radioisotope of technetium (Tc) (e.g., technetium-99m (^{99m}Tc)); e.g., a radioisotope of rhenium (Re) (e.g., rhenium-188 (188Re); e.g., rhenium-186 (186Re)); e.g., a radioisotope of yttrium (Y) (e.g., 90Y); e.g., a radioisotope of lutetium (Lu) (e.g., 177Lu); e.g., a radioisotope of gallium (Ga) (e.g., 68Ga; e.g., 67Ga); e.g., a radioisotope of indium (e.g., 111In); e.g., a radioisotope of copper (Cu) (e.g., 67Cu)] with which 1427 is labelled.

In certain embodiments, 1428 is labelled with (e.g., chelated to) a radioisotope of a metal, to form a compound according to the formula below:

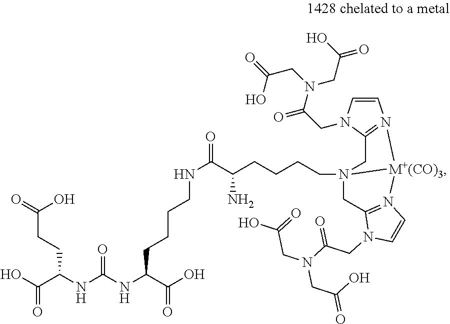

or a pharmaceutically acceptable salt thereof, wherein M is a metal radioisotope [e.g., a radioisotope of technetium (Tc) (e.g., technetium-99m (^{99m}Tc)); e.g., a radioisotope of rhenium (Re) (e.g., rhenium-188 (188Re); e.g., rhenium-186 (186Re)); e.g., a radioisotope of yttrium (Y) (e.g., 90Y); e.g., a radioisotope of lutetium (Lu) (e.g., 177Lu); e.g., a radioisotope of gallium (Ga) (e.g., 68Ga; e.g., 67Ga); e.g., a radioisotope of indium (e.g., 111In); e.g., a radioisotope of copper (Cu) (e.g., 67Cu)] with which 1428 is labelled.

In certain embodiments, a radionuclide labelled PSMA binding agent comprises PSMA I&S:

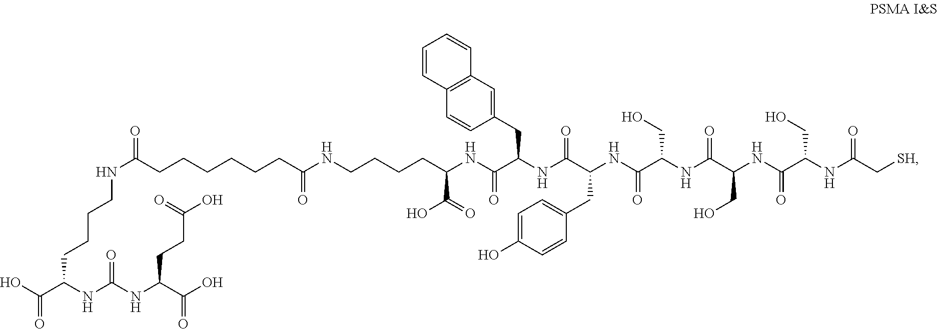 [SMILES: O=C(O)CC[C@H](NC(=O)N[C@@H](CCCCNC(=O)CCCCCCC(=O)NCCCC[C@@H](NC(=O)[C@@H](Cc1ccc2ccccc2c1)NC(=O)[C@@H](Cc1ccc(O)cc1)NC(=O)[C@H](CO)NC(=O)[C@H](CO)NC(=O)[C@H](CO)NC(=O)CS)C(=O)O)C(=O)O)C(=O)O]

or a pharmaceutically acceptable salt thereof. In certain embodiments, a radionuclide labelled PSMA binding agent comprises 99mTc-PSMA I&S, which is PSMA I&S labelled with 99mTc, or a pharmaceutically acceptable salt thereof.

### H. Example Automated PRIMARY Score Determination Illustrative Embodiment

The PRIMARY score staging system is a 5-grade scale. In an illustrative embodiment of the automated scoring method described below, assigning the correct score is based on PET and CT imaging. The CT scan is used to correctly localize the prostate, liver, and aorta within the scan. The PET scans are converted to Standardized Uptake Values (SUV). Further, organ segmentation masks are used to extract relative (corresponding) areas from PET images. Prostate SUV-converted PET scans are analysed by computer software in order to localize uptakes. For each uptake, the peak intensity is localized and the SUV value is measured. The uptake is classified as focal or diffuse. The 3D prostate clinical model is fit to the prostate segmentation mask and the correct prostate zone is assigned to each uptake. Based on the liver and aorta reference SUV values, the PSMA expression score is computed. Having all those data, the proper PRIMARY score is assigned based on the scoring scheme presented in Table 3 (see also FIG. 2 and Table 2 in Emmett et al., showing PRIMARY scoring schemes).

TABLE 3
PRIMARY score staging scheme, proposed in Seifert
et al., European Urology 83 (2023) pp. 405-412.

| | | PSMA | |
|---|---|---|---|
| | | expression | Local tumor |
| PRIMARY score | Pattern and intensity ^{a} | score | (T) extent |
| 1 | No dominant intraprostatic pattern | 0-1 | miT0 |
| | on PSMA. Low grade activity | | |
| 2 | Diffuse transition zone activity or | 1-2 | miT0 |
| | symmetrical central zone activity | | |
| | that does not extend to the prostate | | |
| | margin on CT | | |
| 3 | Focal transition zone activity | 2-3 | miT2, miT3, or |
| | visually twice above background | | miT4 |
| 4 | Focal peripheral zone activity (no | 1-3 | miT2, miT3, or |
| | minimum intensity). | | miT4 |
| 5 | Intense uptake (visual very high | 3 | miT2, miT3, or |
| | intensity or SUVₘₐₓ >12) | | miT4 |

CT = computed tomography;
PSMA = prostate-specific membrane antigen;
SUVₘₐₓ = maximum standardized uptake value.
^{a} Quantitative parameters for the PRIMARY score were established using ⁶⁸Ga-PSMA-11.

Uptakes within the prostate are localized using a hotspot detection algorithm implemented in the aPROMISE methodology described in Seifert et al. and/or Emmet et al. Hotspots below 120 μl are discarded. For each hotspot, the peak position and SUV value are found. Each peak is classified as focal or diffuse. Peaks are also assigned a list of prostate zones, through which they volumetrically expand. The list of prostate zones is sorted in descending order starting from the zone in which a hotspot peak is located and ending in the zone with the least number of hotspot voxels. Moreover, hotspots are checked whether they extend outside the prostate. All this data is then gathered for each single hotspot located within the prostate and a list of uptakes data serves as an input to the PRIMARY score function.

In order for the score to be reproducible, and to standardize its determination, an automated method of its computation was devised and is presented here. In certain embodiments, this PRIMARY score computation technique uses image analysis data automatically identified via one or more CNN models. For example, one or more CNN models can be used for the identification, localization, and quantification of the one or more uptake regions (hotspots) and/or for the identification of the prostate, the zones of the prostate, and/or other organs/regions (e.g., the liver and the aorta) as they appear in the anatomical image (e.g., CT, X-ray, or MRI image) and/or as those regions are mapped to the 3D functional image (e.g., the 3D PSMA-PET image).

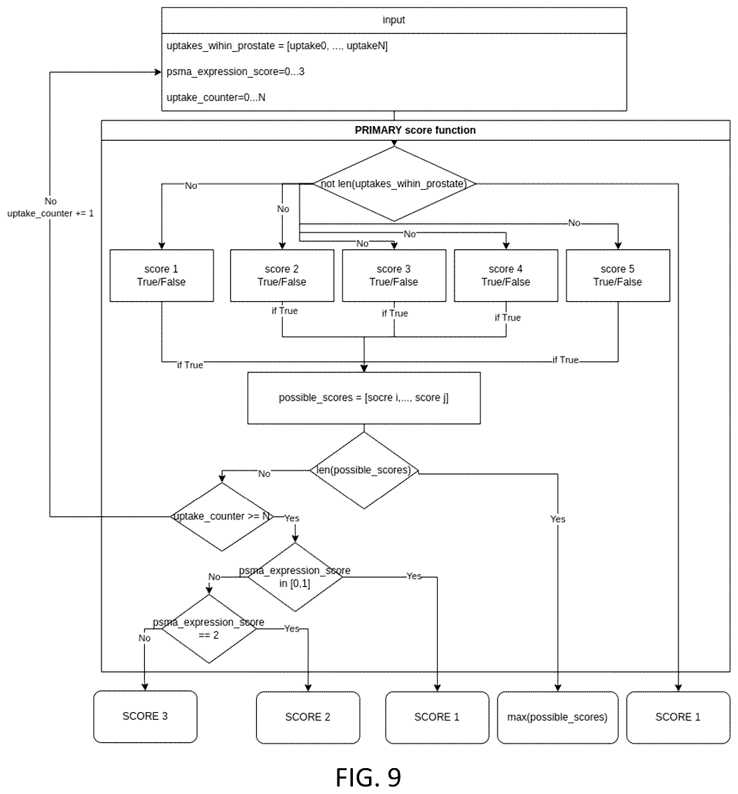

For PRIMARY score automatic computation, a Python script was written (see illustrative pseudocode excerpt below). A schematic diagram of this illustrative method is presented in FIG. 9. In this illustrative example, the input to the function is a list of uptakes (hotspots data), PSMA Expression score, and an uptake counter. The uptake counter indicates which prostate zone from the prostate zones list for a single hotspot to use for the prostate zone evaluation criteria. It is common for a single hotspot to expand through multiple prostate zones. Usually, hotspots are evaluated based on the hotspot peak location zone. However, it might happen that no score matches the peak location prostate zone. In that case, the function is recurrently called, with the indication to pick the second, third, and further prostate zones for the evaluation criteria. The score is evaluated then not on the hotspot peak location prostate zone, but on the following prostate zones through which the hotspot expands. The uptake counter is progressively increased until it is possible to assign at least one PRIMARY score to the patient. It may happen that after checking all prostate zones through which hotspots expand, it is still not possible to assign any PRIMARY score to the patient. In case all iterations fail to find the primary score, the score is assigned based on the PSMA Expression score only. If the PSMA Expression score is 0 or 1, the PRIMARY score 1 is assigned. If the PSMA Expression score is 2, the PRIMARY score of 2 is assigned. Otherwise, the PRIMARY score of 3 is assigned.

Since a subject may have multiple prostate localized uptakes, it is also possible that multiple scores can be assigned to a single patient. In that case, the worst score scenario is chosen.

Pseudocode: Illustrative pseudocode excerpt
for automated PRIMARY score computation

```
□func compute_score(
  uptakes_within_prostate = [uptake0, ...uptakeN],
  psma_expression_score=0...3,
  uptake_prostate_zone_cnt=0...N,
)
  if len(uptakes_within_prostate) == 0:
        return SCORE1
  score1 = score_1(
        uptakes_within_prostate,
        psma_expression_score,
        uptake_prostate_zone_cnt,
  )
  score2 = score_2(
        uptakes_within_prostate,
        psma_expression_score,
        uptake_prostate_zone_cnt,
    )
    score3 = score_3(
          uptakes_within_prostate,
          psma_expression_score,
          uptake_prostate_zone_cnt,
    )
    score4 = score_4(
          uptakes_within_prostate,
          psma_expression_score,
          uptake_prostate_zone_cnt,
    )
    score5 = score_5(
          uptakes_within_prostate,
          psma_expression_score,
          uptake_prostate_zone_cnt,
    )
    primary_scores = [score1,score2,score3,score4,score5]
    possible_primary_scores = [ ]
    for idx, score in enumerate(primary_scores):
          if score:
              possible_primary_score.append(idx+1)
    if (not possible_primary_scores) and
          (uptake_prostate_zone_cnt = N):
          uptake_prostate_zone_cnt += 1
          possible_primary_scores = compute_score(
              uptakes_within_prostat,
              psma_expression_score,
              uptake_prostate_zone_cnt,
          )
    if not possible_primary_scores:
          if psma_expression_score in [0,1]:
              return 1
          elif psma_expression_score == 2:
                return 2
            else: return 3
      return max(possible_primary_scores)
```

The technique requires multiple indirect pre-computed variables to correctly assign the score, among others: uptake localization within the prostate, uptake mask, uptake type classification as diffuse or focal, prostate zones (transition, peripheral, central), and PSMA expression score. Illustrative examples of the computation of those variables are presented in detail in the sections below.

TABLE 4
Example Score Categories Used in an Automated
Prostate Cancer Staging Scoring Method

| | |
|---|---|
| Score 1 | The score 1 is assigned if the PSMA Expression score is equal to 0 or 1 and |
| | there is no focal uptake within the prostate. The score 1 is also assigned in |
| | cases where no other score can be assigned and the PSMA Expression score is |
| | equal to 0 or 1. |
| Score 2 | The score 2 is assigned if there is a diffuse uptake in the transition zone or any |
| | kind of uptake in a central zone, and the PSMA Expression score is 1 or 2. |
| Score 3 | The score 3 is assigned if the PSMA Expression score is 2 or 3 and there is a |
| | focal uptake in the prostate transition zone. |
| Score 4 | The score 4 is assigned if the PSMA Expression score is 1, 2, or 3 and there is a |
| | focal uptake in the peripheral zone. |
| Score 5 | The score 5 is assigned if the PSMA Expression score is 3 and the highest |
| | prostate-located peak SUV value is above 12. |

### H.i. Standardized Uptake Value (SUV)

In certain embodiments, the PET image is converted to SUV according to the following equation:

$$pet\_suv = ( ( pet\_image + shift ) * slope ) + intercept \qquad ( 6 )$$

where shift, slope, and intercept are parameters computed based on the subject weight, injection dose, and other PET scan-related variables.

### H.ii. Uptake Peak Localization and Peak SUV Value Calculation

In certain embodiments, for the purpose of extracting uptake regions, the hotspot detection algorithm implemented in aPROMISE is used. Only prostate-located hotspots (hotspots that have an intersection with the prostate segmentation mask) are taken. Hotspots are filtered by their volume. Hotspots below 120 μl are discarded.

The peak position is the maximum SUV value within the hotspot and prostate area:

$${position}_{peak} = ar \text{?} ( {hotspot}_{mask} ⋃ {prostate}_{mask} ) \qquad ( 7 )$$

$$\text{?} \text{indicates text missing or illegible when filed}$$

where hotspot_mask is a boolean mask (e.g., for a single hotspot) and prostate_mask is filled with prostate SUV values.

The peak value is the mean SUV value in a close location to the peak position. The cube with a 3-voxel side is centered on the peak position. The mean value of voxels belonging to the hotspot and within the cube is computed.

$$( 8 )$$

$${peak}_{value} = mean ( {prostate}_{suv} [ {position}_{peak} - 1 : {position}_{peak} + 1 ] * {hotspot}_{mask} )$$

### H.iii Uptake Classification

Each prostate-located uptake is assigned a diffuse or focal classification label. If the peak value is greater than twice the value of the prostate_SUV_mean, then the uptake is classified as focal, otherwise diffuse.

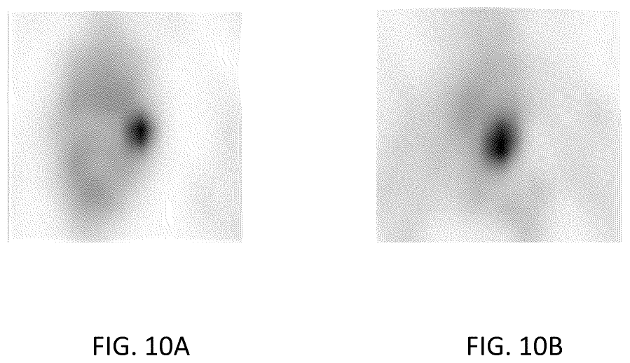

FIGS. 10A and 10B are example images of prostate-located uptake regions (hotspots) that are classified as “diffuse” and “focal”, respectively, according to an illustrative embodiment.

### H.iv Prostate Zones

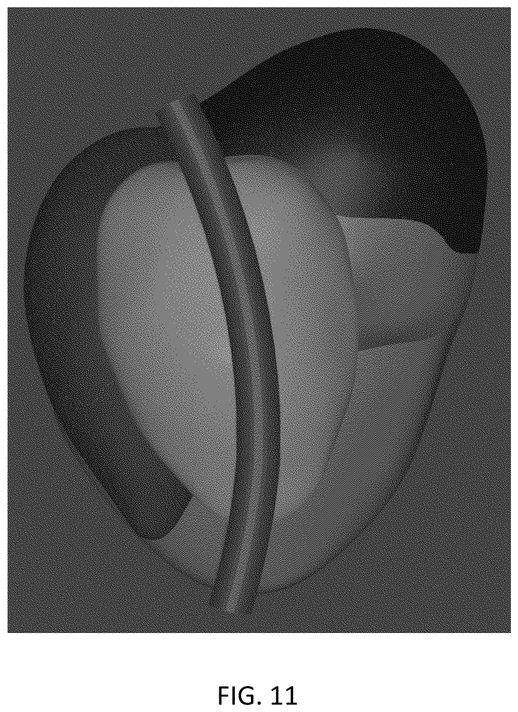

In order to identify the one or more prostate zones for each of the uptake regions (hotspots), a prostate clinical model is fit to the prostate in the CT image. The prostate clinical model was developed as a stl 3D model, converted to numpy array object, and loaded in Python. The 3D prostate model is presented in FIG. 11.

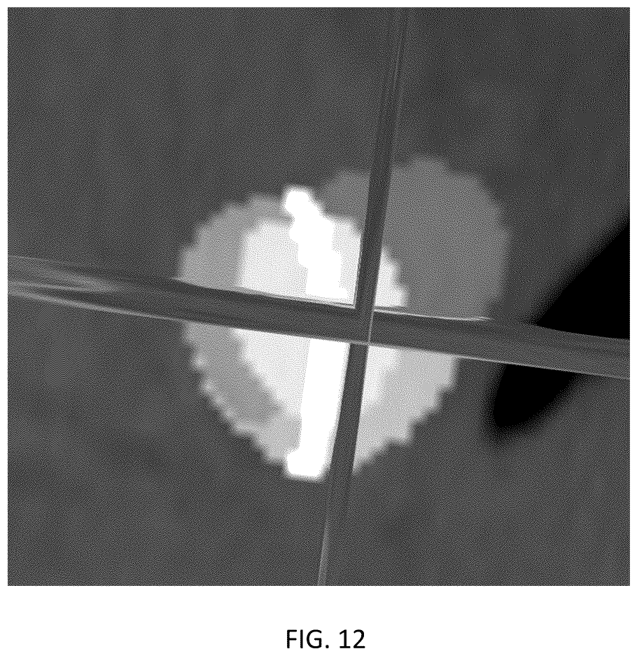

The fitting procedure is done using a prostate segmentation mask generated by the aPROMISE algorithm, an example of which is illustrated in FIG. 12. The prostate model is assumed to have the correct orientation, only the scale and position are adjusted. The prostate segmentation mask size is computed, as the distance between the furthest prostate points in the x, y, and z axes. The same method is applied to the 3D prostate model size measurement. The scale is computed as the ratio of two measurements and the prostate 3D model is scaled accordingly.

The position of the 3D prostate model is calculated in a very similar way, to how the scale is computed. The furthest prostate points in the x, y, and z axes are assumed to have the same coordinates in the case of the prostate segmentation mask and the 3D prostate model.

As both PET and CT images are aligned together and have the same voxel size, the 3D prostate model can be directly applied to the PET image.

For each hotspot, the list of prostate zones is assigned, through which the hotspot expands. The list is sorted in descending order, excluding the first element. The first element in the list is always the prostate zone, in which the uptake peak is located. All other elements are sorted in descending order based on the number of voxels within the zone. Each uptake can thus have a list of 1 to 5 elements (central, fibromuscular, peripheral, transition, and ureter zone). The fibromuscular zone and ureter zone are not included in the PRIMARY score staging system described in Emmett et al. Accordingly, if scoring is based directly on the system described in Emmett et al., in certain cases, no score may be assigned to the patient. For example, if there is only a single focal uptake in a fibromuscular zone, a PRIMARY score exactly corresponding to the 5 grade scale described would not be assigned initially. In that case, the PRIMARY score is assigned based on the expression score, according to the formula presented in the Automated Primary Score Determination section above. In certain cases, a score cannot be assigned to the patient based on the uptake peak location prostate zone. This can be the case, for example, if the uptake peak is located in the fibromuscular zone and there are no other prostate located uptakes. However, if the hotspot expands to other prostate zones, the score could be assigned by verifying the PRIMARY score conditions for the other prostate zones through which the uptake extends. In that case, the following prostate zone from the hotspots prostate zones list is taken, and treated as the main prostate zone for an uptake in a staging system.

### H.v PSMA Expression Score

The PSMA Expression score is assigned based on the PROMISE V2 staging system proposed in Seifert et al., “Second Version of the Prostate Cancer Molecular Imaging Standardized Evaluation Framework Including Response Evaluation for Clinical Trials (PROMISE V2),” European Urology 83 (2023) pp. 405-412. In the first step, the aorta SUV mean value and liver SUV mean value is computed based on the organ segmentation masks and SUV-converted PET scan. The highest uptake peak within the prostate-i.e., among all identified prostate hotspots, the PSMA expression score was computed based on the hotspot with the highest peak update. The highest peak uptake value was compared with the aorta and liver SUV mean values, and the PSMA Expression score is assigned based on the conditions presented in Table 5 below. PSMA expression score, accordingly, was determined as a lesion index in line with the approach illustrated in FIG. 5A, herein (i.e., a step-wise, discrete scoring approach), but other approaches are possible, such as the lesion index scoring approach shown in FIG. 5B.

TABLE 5
PSMA Expression score staging system, based on PROMISE v2 framework.

| | Reported | | | PSMA status for |
|---|---|---|---|---|
| | PSMA | Uptake (PROMISE | Uptake (PROMISE | PSMA radioligand |
| Score | expression | V1) | V2) | therapy^{a} |
| 0 | No | Below blood pool | Equal to or lower | Negative |
| | | | than blood pool | |
| 1 | Low | Equal to or above | Equal to or lower | Negative |
| | | blood pool and | that liver^{b} and | |
| | | lower than liver^{b} | higher than blood | |
| | | | pool | |
| 2 | Intermediate | Equal to or above | Equal to or lower | Positive |
| | | liver^{b} and lower | than parotid gland | |
| | | than parotid gland | and higher than | |
| | | | liver^{b} | |
| 3 | High | Equal to or above | Higher than parotid | Positive |
| | | parotid gland | gland | |

PROMISE = prostate cancer molecular imaging standardized evaluation; PSMA = prostate-specific membrane antigen.
^{a}For detailed criteria of selecting patients for PSMA radioligand therapy including lesion size and nature of lesions (lymph node, bone, and visceral), see the work of Kuo et al.
^{b}For PSMA ligands with liver dominant excretion (e.g., [¹⁸F]F-PSMA1007), the spleen is recommended as the reference organ instead of the liver.

### H.vi. PRIMARY Scores Assignment Example

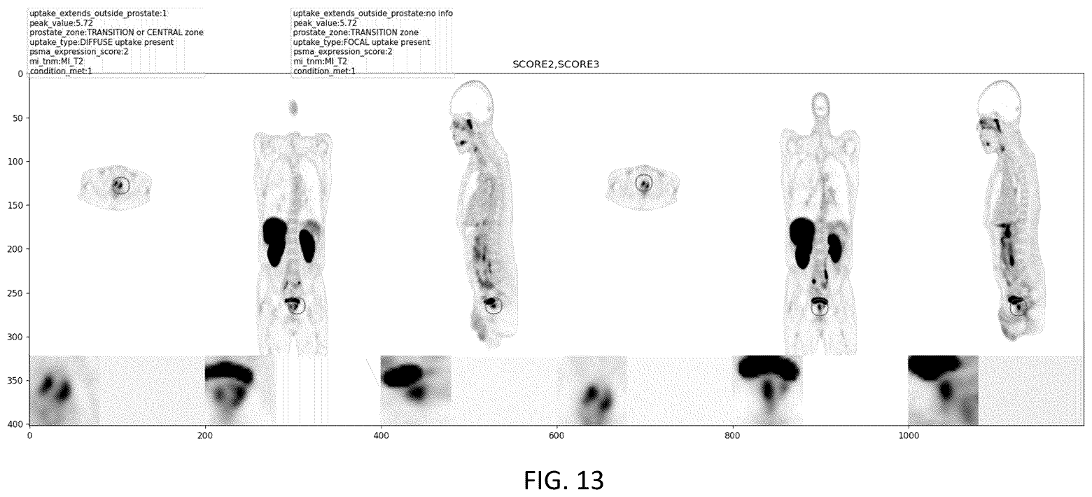

FIG. 13 is a compilation of images depicting an exemplary automated computation of a PRIMARY score, according to an illustrative embodiment. Here, the patient has two prostate-located uptakes-one diffuse uptake located in the prostate transition zone, and one focal uptake located in the prostate transition zone as well. The PSMA Expression score for the patient is equal to 2. Having this information, the PRIMARY score of 2 and 3 is assigned. The algorithm takes the worst-case scenario, so the PRIMARY score of 3 is the final algorithm prediction.

### EQUIVALENTS

In some implementations, various modules described herein can be separated, combined or incorporated into single or combined modules. Modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

While the invention has been particularly shown and described with reference to specific preferred embodiments, it should be understood by those skilled in the art that various changes in form and detail may be made therein without departing from the spirit and scope of the invention as defined by the appended claims.

1-17. (canceled)

18. A method for automated determination of a prostate cancer staging score for a subject, the method comprising:
(a) receiving, by a processor of a computing device, a 3D functional image of the subject;
(b) determining, by the processor, a prostate volume within the 3D functional image, said prostate volume identifying a region of the 3D functional image corresponding to a prostate of the subject;
(c) localizing, by the processor, one or more uptake regions within the 3D functional image, each determined to represent a lesion or potential lesion within the prostate of the subject or a vicinity thereof;
(d) determining, by the processor, for each particular uptake region of the one or more uptake regions:
(i) values of one or more uptake region intensity metrics, each corresponding to a measure of intensity within and/or characteristic of the particular uptake region; and
(ii) a set of assigned prostate zones identifying, for the particular uptake region, one or more spatial zones within or about the prostate of the subject that the particular uptake region is associated with; and
(e) determining, by the processor, the prostate cancer staging score based at least in part on (i) the values of the one or more intensity metrics and (ii) the set of assigned prostate zones determined for the one or more uptake regions.

19-35. (canceled)

36. A system for automated determination of a prostate cancer staging score for a subject, the system comprising:
a processor of a computing device; and
memory having instructions stored thereon, wherein the memory, when executed by the processor, causes the processor to:
(a) receive a 3D functional image of the subject;
(b) determine a prostate volume within the 3D functional image, said prostate volume identifying a region of the 3D functional image corresponding to a prostate of the subject;
(c) localize one or more uptake regions within the 3D functional image, each determined to represent a lesion or potential lesion within the prostate of the subject or a vicinity thereof;
(d) determine, for each particular uptake region of the one or more uptake regions:
(i) values of one or more uptake region intensity metrics, each corresponding to a measure of intensity within and/or characteristic of the particular uptake region; and
(ii) a set of assigned prostate zones identifying, for the particular uptake region, one or more spatial zones within or about the prostate of the subject that the particular uptake region is associated with; and
(e) determine the prostate cancer staging score based at least in part on (i) the values of the one or more intensity metrics and (ii) the set of assigned prostate zones determined for the one or more uptake regions.

37. The method of claim 18, wherein the set of assigned prostate zones identified for each of the one or more uptake regions are selected from a set of possible prostate zones, said set of possible prostate zones comprising one or more of (A), (B), and (C) as follows:
(A) a central zone surrounding ejaculatory ducts and comprising about 25% of a prostate total mass,
(B) a transition zone comprising a portion of the prostate surrounding a urethra, and
(C) a peripheral zone situated toward a back of the prostate and comprising a majority of prostate tissue.

38. The method of claim 37, wherein the set of possible prostate zones further comprises a fibromuscular zone and/or a ureter zone.

39. The method of claim 18, wherein the one or more uptake regions are hotspots.

40. The method of claim 18, wherein the 3D functional image is a three-dimensional (3D) positron emission tomography (PET) image of the subject obtained following administration to the subject of a radiopharmaceutical comprising a prostate-specific membrane antigen (PSMA) binding agent.

41. The method of claim 40, wherein the PSMA binding agent comprises [¹⁸F]DCFPyL.

42. The method of claim 40, wherein the PSMA binding agent comprises ⁶⁸Ga-PSMA-11.

43. The method of claim 18, wherein the one or more uptake region intensity metrics comprise a peak uptake region intensity.

44. The method of claim 18, comprising, determining, by the processor, for each particular uptake region of the one or more uptake regions, a corresponding uptake classification label indicative of whether the particular uptake region is focal or diffuse and, at step (e) using the uptake classification labels determined for the one or more uptake regions to determine the prostate cancer staging score.

45. The method of claim 18, wherein determining the set of assigned prostate zones for each particular uptake region of the one or more uptake regions comprises, (i) sorting a list of prostate zones in descending order starting from a zone in which a peak of the particular uptake region is located and ending in a zone with a least number voxels of the uptake region, and (ii) identifying whether the uptake region extends outside the prostate.

46. The method of claim 18, comprising:
localizing, by the processor, within the 3D functional image, a liver volume and/or an aorta volume; and
determining, by the processor, one or more liver reference intensities for a liver and/or one or more aorta reference intensities for an aorta, each corresponding to a measure of intensity within and/or characteristic of uptake in the liver volume and/or the aorta volume, respectively.

47. The method of claim 46, comprising determining a lesion index value based on (i) the one or more uptake region intensity metrics and (ii) the one or more uptake intensity metrics for the liver and/or the one or more uptake intensity metrics for the aorta.

48. The method of claim 18, comprising, at step (b):
using one or more machine learning module(s) implementing convolutional neural networks (CNNs) to segment a 3D anatomical image and generate a 3D segmentation map that identifies a 3D boundary of a prostate representation within the 3D anatomical image; and
transferring the 3D segmentation map to the 3D functional image to localize the prostate volume therein.

49. The method of claim 18, comprising, at step (c), localizing the one or more uptake regions within the 3D functional image using one or more machine learning module(s).

50. The system of claim 36, wherein the set of assigned prostate zones identified for each of the one or more uptake regions are selected from a set of possible prostate zones, said set of possible prostate zones comprising one or more of (A), (B), and (C) as follows:
(A) a central zone surrounding ejaculatory ducts and comprising about 25% of a prostate total mass,
(B) a transition zone comprising a portion of the prostate surrounding a urethra, and
(C) a peripheral zone situated toward a back of the prostate and comprising a majority of prostate tissue.

51. The system of claim 50, wherein the set of possible prostate zones further comprises a fibromuscular zone and/or a ureter zone.

52. The system of claim 36, wherein the one or more uptake regions are hotspots.

53. The system of claim 36, wherein the 3D functional image is a three-dimensional (3D) positron emission tomography (PET) image of the subject obtained following administration to the subject of a radiopharmaceutical comprising a prostate-specific membrane antigen (PSMA) binding agent.

54. The system of claim 36, wherein the instructions cause the processor to, at step (b):
use one or more machine learning module(s), implementing convolutional neural networks (CNNs), to segment a 3D anatomical image and generate a 3D segmentation map that identifies a 3D boundary of a prostate representation within the 3D anatomical image; and
transfer the 3D segmentation map to the 3D functional image to localize the prostate volume therein.
